(19) United States
(12) Patent Application Publication
(10) Pub. No.: US 2002/0082426 A1
(43) Pub. Date: Jun. 27, 2002

(54) Protease inhibitors

(75) Inventors: Ashley E. Fenwick, Hertfordshire (GB); Andrew D. Gribble, Knebworth (GB); Robert W. Marquis JR., St. Davids, PA (US); Jason Witherington, Hertfordshire (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/083,910

(22) Filed: Feb. 27, 2002

Related U.S. Application Data
(63) Continuation of application No. 09/719,342, filed on Dec. 11, 2000, now abandoned.
Application No. 09/719,342 is a 371 of international application No. PCT/US99/13334, filed on Jun. 11, 1999.
(60) Provisional application No. 60/089,056, filed on Jun. 12, 1998.

(51) Int. Cl. C 07D 4 1/02; A61K 31/4439; A61K 31/4015

(52) U.S. Cl. 546/276.4; 548/543; 514/424; 514/343

(57) ABSTRACT

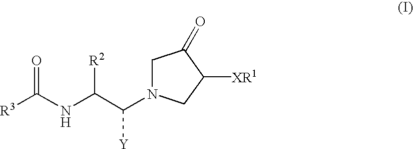

This invention relates to compounds of formula (I) or a pharmaceutically acceptable salt thereof, which are inhibitors of cysteine proteases, particularly cathepsin K, and are useful in the treatment of diseases in which inhibition of bone loss is a factor.

### FIELD OF THE INVENTION

This invention relates to novel protease inhibitors, particularly inhibitors of cysteine and serine proteases, more particularly compounds which inhibit cysteine proteases. The compounds of this invention even more particularly relate to those compounds which inhibit cysteine proteases of the papain superfamily, and particularly cysteine proteases of the cathepsin family. In the most preferred embodiment, this invention relates to compounds which inhibit cathepsin K. Such compounds are particularly useful for treating diseases in which cysteine proteases are implicated, especially diseases of excessive bone or cartilage loss, e.g., osteoporosis, periodontitis, and arthritis.

### BACKGROUND OF THE INVENTION

Cathepsin K is a member of the family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard, M. J., et al., (1996) J. Biol. Chem. 271, 12517-12524; Drake, F. H., et al., (1996) J. BioL Chem. 271, 12511-12516; Bromme, D., et al., (1996) J. Biol Chem. 271, 2126-2132.

Cathepsin K has been variously denoted as cathepsin O, cathepsin X or cathepsin O2 in the literature. The designation cathepsin K is considered to be the more appropriate one (name assigned by Nomenclature Committee of the International Union of Biochemistry and Molecular Biology).

Cathepsins of the papain superfamily of cysteine proteases function in the normal physiological process of protein degradation in animals, including humans, e.g., in the degradation of connective tissue. However, elevated levels of these enzymes in the body can result in pathological conditions leading to disease. Thus, cathepsins have been implicated in various disease states, including but not limited to, infections by pneumocystis carinii, trypsanoma cruzi, trypsanoma brucei brucei, and Crithidia fusiculata; as well as in schistosomiasis malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Cathepsin K is believed to play a causative role in diseases of excessive bone or cartilage loss. Bone is composed of a protein matrix in which spindle- or plate-shaped crystals of hydroxyapatite are incorporated. Type I Collagen represents the major structural protein of bone comprising approximately 90% of the structural protein. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodeling at discrete foci throughout life. These foci, or remodeling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

It now has been discovered that a novel class of compounds are protease inhibitors, most particularly inhibitors of cathepsin K, and these compounds are useful for treating diseases in which inhibition of bone resorption is indicated, such as osteoporosis and periodontal disease.

### SUMMARY OF THE INVENTION

An object of the present invention is to provide protease inhibitors, such as inhibitors of cysteine and serine proteases. In particular, the present invention relates to compounds which inhibit cysteine proteases, and particularly cysteine proteases of the papain superfamily. Preferably, this invention relates to compounds which inhibit cysteine proteases of the cathepsin family and particularly, compounds which inhibit cathepsin K. The compounds of the present invention are useful for treating diseases, which may be therapeutically modified by altering the activity of such proteases.

Accordingly, in the first aspect, this invention provides a compound according to formula (I).

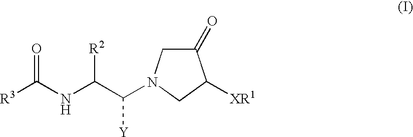

In another aspect, this invention provides a pharmaceutical composition comprising a compound according to formula (I) and a pharmaceutically acceptable carrier.

In yet another aspect, this invention provides a method of treating diseases in which the disease pathology may be therapeutically modified by inhibiting proteases, such as cysteine and serine proteases. In particular, the method includes treating diseases by inhibiting cysteine proteases, and particularly cysteine proteases of the papain superfamily. More particularly, the inhibition of cysteine proteases of the cathepsin family, such as cathepsin K is described.

In another aspect, the compounds of this invention are especially useful for treating diseases characterized by bone loss, such as osteoporosis and gingival diseases, such as gingivitis and periodontitis, or by excessive cartilage or matrix degradation, such as osteoarthritis and rheumatoid arthritis.

In yet another aspect of this invention, this invention provides a method of producing the compounds having the formula (I) above.

### DETAILED DESCRIPTION OF THE INVENTION

The present invention provides alkoxypyrrolidinone compounds of formula (I):

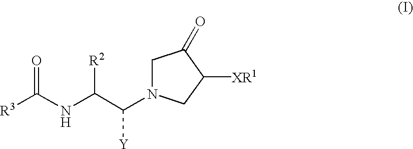

wherein:

X is selected from the group consisting of oxygen, sulfur, SO, and SO₂;

Y is selected from the group consisting of H₂ and oxygen; where if Y is H₂, then the - - - bond represents two single bonds and where if Y is O, then the - - - bond represents a double bond;

R¹ is selected from the group consisting of hydrogen, C₁₋₆ alkyl, C₂₋₆ alkenyl, C₂₋₆ alkynyl, C₃₋₆ cycloalkyl-C₀₋₆ alkyl, Ar—C₀₋₆ alkyl, Het-C₀₋₆ alkyl, (CH₂)₀₋₆CO₂R″, and (CH₂)₀₋₆Ar;

R² is selected from the group consisting of H, C₁₋₆ alkyl, C₂₋₆ alkenyl, C₂₋₆ alkynyl, C₃₋₆ cycloalkyl-C₀₋₆ alkyl, Ar—C₀₋₆ alkyl, and Het-C₀₋₆ alkyl;

R³ is selected from the group consisting of H, C₁₋₆ alkyl, C₂₋₆ alkenyl, C₂₋₆ alkynyl, C₃₋₆ cycloalkyl-C₀₋₆ alkyl, Ar—C₀₋₆ alkyl, and Het-C₀₋₆ alkyl;

or pharmaceutically acceptable salts, hydrates, and isomers thereof.

Preferably, X is O.

Preferably, Y is O.

Preferably, R¹ is selected from the group consisting of

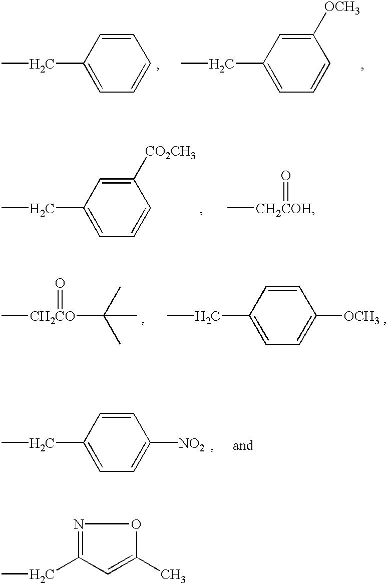

Suitably, R² is isobutyl or a substituted isobutyl.

Suitably, R³ is selected from the group consisting of

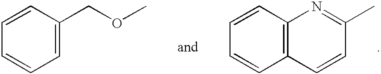

Suitably, R″ is selected from the group consisting of hydrogen, C₁₋₆ alkyl, C₂₋₆ alkenyl, C₂₋₆ alkynyl, C₃₋₆ cycloalkyl-C₀₋₆ alkyl, Ar-C₀₋₆ alkyl, and Het-C₀₋₆ alkyl;

The present invention includes all hydrates, solvates, complexes and prodrugs of the compounds of this invention. Prodrugs are any covalently bonded compounds which release the active parent drug according to formula (I) in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in formula (I) or any subformula thereof is independent of its meaning, or any other substituent's meaning, at any other occurrence, unless specified otherwise.

Specific representative compounds of this invention include:

3-Benzyloxy-1-(N-carbobenzyloxy-L-leucyl)pyrrolidin-4-one;

3-Benzylthio-1-(N-carbobenzyloxy-L-leucyl)pyrrolidin-4-one;

3-Benzylsulfinyl-1-(N-carbobenzyloxy-L-leucyl)pyrrolidin-4-one;

3-Benzylsulfonyl-1-(N-carbobenzyloxy-L-leucyl)pyrrolidin-4-one;

1-[2-(Benzyloxycarbonylamino)-4-methylpentyl]-3-benzylthiopyrrolidin-4-one;

3-Benzylthio-1-[N-(2-quinolinecarbonyl)-L-leucyl]pyrrolidin-4-one;

3-tert-Butoxycarbonylmethoxy-1-(N-carbobenzyloxy-L-leucyl)pyrrolidin-4-one;

3-(3-Methoxybenzyloxy)-1-(N-carbobenzyloxy-L-leucyl)pyrrolidin-4-one;

3-(3-Methoxycarbonylbenzyloxy)-1-(N-carbobenzyloxy-L-leucyl)pyrrolidin-4-one;

3-tert-Butoxycarbonylmethoxy-1-[N-(2-quinolinecarbonyl)-L-leucyl]pyrrolidin-4-one;

1-[N-(2-Quinotinecarbonyl)-L-leucyl]-3-oxo-4-pyrrolidineooxyacetic acid;

3-(3-Methoxybenzyloxy)-1-[N-(2-quinolinecarbonyl)-L-leucyl]pyrrolidin-4-one;

3-(4-Methoxybenzyloxy)-1-[N-(2-quinolinecarbonyl)-L-leucyl]pyrrolidin-4-one;

3-(3-Methoxycarbonylbenzyloxy)-1-[N-(2-quinolinecarbonyl)-L-leucyl]pyrrolidin-4-one;

3-(4-Nitrobenzyloxy)-1-[N-(2-quinolinecarbonyl)-L-leucyl]pyrrolidin-4-one;

3-(5-Methyl-3-isoxazolylmethoxy)-1-[N-(2-quinolinecarbonyl)-L-leucyl]pyrrolidin-4-one;

3-(3-Methoxybenzyloxy)-1-[N-(2-naphthalenecarbonyl)-L-leucyl]pyrrolidin-4-one; and

3-(4-Methoxyphenoxy)-1-(N-carbobenzyloxy-L-leucyl)pyrrolidin-4-one and pharmaceutically acceptable salts thereof.

In yet another aspect, this invention provides novel intermediates useful in the preparation of formula (I) compounds represented by:

3-Benzyloxy-4-hydroxy-1-(N-carbobenzyloxy-L-leucyl)pyrrolidine;

3-Benzylthio-4-hydroxy-1-(N-carbobenzyloxy-L-leucyl)pyrrolidine;

1-[2-(Benzyloxycarbonylamino)-4-methyl-pentyl]-3-benzylthio-4-hydroxypyrrolidine;

3-Benzylthio-4-hydroxy-1-[N-(2-quinolinecarbonyl)-L-leucyl]pyrrolidine;

3-tert-Butoxycarbonylmethoxy-4-hydroxy-1-(N-carbobenzyloxy-L-leucyl)pyrrolidine;

3-Hydroxy-4-(3-methoxybenzyloxy)-1-(N-carbo-benzyloxy-L-leucyl)pyrrolidine;

3-Hydroxy-4-(3-methoxy-carbonylbenzyloxy)-1-(N-carbobenzyloxy-L-leucyl)pyrrolidine;

3-tert-Butoxycarbonylmethoxy-1-[N-(2-quinolinecarbonyl)-L-leucyl]pyrrolidine;

3-Hydroxy-4-(3-methoxycarbonylbenzyloxy)-1-[N-(2-quinotinecarbonyl)-L-leucyl]pyrrolidine;

3-Hydroxy-4-(4-nitrobenzyloxy)-1-[N-(2-quinolinecarbonyl)-L-leucyl]pyrrolidine;

3-Hydroxy-4(5-Methyl-3-isoxazolylmethoxy)-1-[N-(2-quinotinecarbonyl)-L-leucyl]pyrrolidine;

3-Hydroxy-4-(3-methoxybenzyloxy)-1-[N-(2-naphthalenecarbonyl)-L-leucyl]pyrrolidine;

3-Hydroxy-4-(4-methoxyphenoxy)-1-(N-carbobenzyloxy-L-leucyl)pyrrolidine;

1-(N-Phthaloyl-L-leucyl)-3-pyrroline; and

3,4-Dihydroxy-1-(N-phthaloyl-L-leucyl)pyrrolidine)

or salts thereof.

These intermediates are prepared using methods analogous to that described in Scheme 1, Scheme 2, Scheme 3 and the Examples described hereinafter.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984). The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

Certain radical groups are abbreviated herein. t-Bu refers to the tertiary butyl radical; Boc or BOC refers to the t-butyloxycarbonyl radical; Fmoc refers to the fluorenylmethoxycarbonyl radical; Ph refers to the phenyl radical; and Cbz or CBZ or Z refers to the benzyloxycarbonyl radical.

Certain reagents are abbreviated herein. DCC refers to dicyclohexylcarbodiimide; EDC or EDCI refers to N-ethyl-N′(dimethylaminopropyl)-carbodiimide; HOBT or HOBt refers to 1-hydroxybenzotriazole; DMF refers to dimethyl formamide; DIEA refers to di-isopropylethylamine; HoAt refers to 1-hydroxy-7-aza-benzotriazote; Dess-Martin's reagent is 1,1,1-triacetoxy-1,1-dihydro-1,2-benziodoxol-3(1H)-one; TFA refers to trifluoroacetic acid; and THF refers to tetrahydrofuran. “C₁₋₆ alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. Any C₁₋₆alkyl group may be optionally substituted independently by one or two halogens, SR′, OR′, N(R′)₂, C(O)N(R′)₂, carbamyl or C₁₋₄alkyl, where R′ is H or C₁₋₆alkyl. C₀alkyl means that no alkyl group is present in the moiety. Thus, Ar-C₀alkyl is equivalent to Ar. “C₃₋₆ cycloalkyl” as applied herein is meant to include substituted [i.e., alkyl, OR, SR or halogen) and unsubstituted cyclopropane, cyclobutane, cyclopentane, and cyclohexane. “C₂₋₆ alkenyl” as applied herein means an alkyl group of 2 to 6 carbons, wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included. “C₂₋₆ alkynyl” means an alkyl group of 2 to 6 carbons, wherein one carbon-carbon single bond is replaced by a carbon-carbon triple bond. C₂₋₆ alkynyl includes acetylene, 1-propyne, 2-propyne, 1-butyne, 2-butyne, 3-butyne, and the simple isomers of pentyne and hexyne. “Ar” or “aryl” means unsubstituted phenyl or naphthyl; or phenyl or naphthyl substituted by one or more of Ph-C₀₋₆ alkyl, Het-C₀₋₆ alkyl, C₁₋₆ alkoxy, Ph-C₀₋₆ alkoxy, Het-C₀₋₆ alkoxy, OH, (CH₂)₀₋₆CO₂R″, where R″ is as defined above, (CH₂)₁₋₆N R′R′, O(CH₂)₁₋₆NR′R′; wherein each R′ independently is H, C₁₋₆ alkyl, Ar-C₀₋₆ alkyl, or Het-C₀₋₆ alkyl; or phenyl or naphthyl substituted by one to three moieties selected from C₁₋₄alkyl, OR′, N(R′)₂, SR′, CF₃, NO₂, CN, CO₂R′, CON(R′), F, Cl, Br and I, or substituted by a methylenedioxy group.

As used herein “Het” or “heterocyclic” represents a stable 5- to 7-membered monocyclic or a stable 7- to 10-membered bicyclic heterocyclic ring, which is either saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. and may optionally be substituted with one or two moieties selected from C₁₋₄alkyl, OR′, N(R′)₂, SR′, CF₃, NO₂, CN, CO₂R′, CON(R′), F, Cl, Br and I, where R′ is as defined hereinbefore. Examples of such heterocycles include piperidinyl, piperazinyl 2-oxopiperazinyl, 2-oxopiperidinyl, 2-oxopyrrolodinyl, 2-oxoazepinyl, azepinyl, thienyl, pyrrolyl, 4 piperidonyl, pyrrolidinyl, pyrazolyl, pyrazolidinyl, imidazolyl, pyridyl, pyrazinyl, oxazolidinyl, oxazolinyl, oxazolyl, isoxazolyl, morpholinyl, thiazolidinyl, thiazolinyl, isothiazolyl, thiazolyl, quinuclidinyl, indolyl, quinotinyl, isoquinolinyl, benzimidazolyl, benzothienyl, benzopyranyl, benzoxazolyl, benzofuranyl, furyl, pyranyl, tetrahydrofuryl, tetrahydropyranyl, thienyl, benzoxazolyl , thiamorpholinyl sulfoxide, thiamorpholinyl sulfone, oxadiazolyl, benzothiazolyl, benzoisothiazolyl, benzisoxazolyl, pyrimidinyl, cinnolinyl, quinazolinyl, quinoxalinyl, 1,5-napthyridinyl, 1,6-napthyridinyl, 1,7-napthyridinyl, 1,8-napthyridinyl, tetrazolyl, 1,2,3-triazolyl, and 1,2,4-triazolyl. Compounds of the formula (I) are prepared by methods analogous to those described in the solution synthesis method of Scheme 1, 2 or 3.

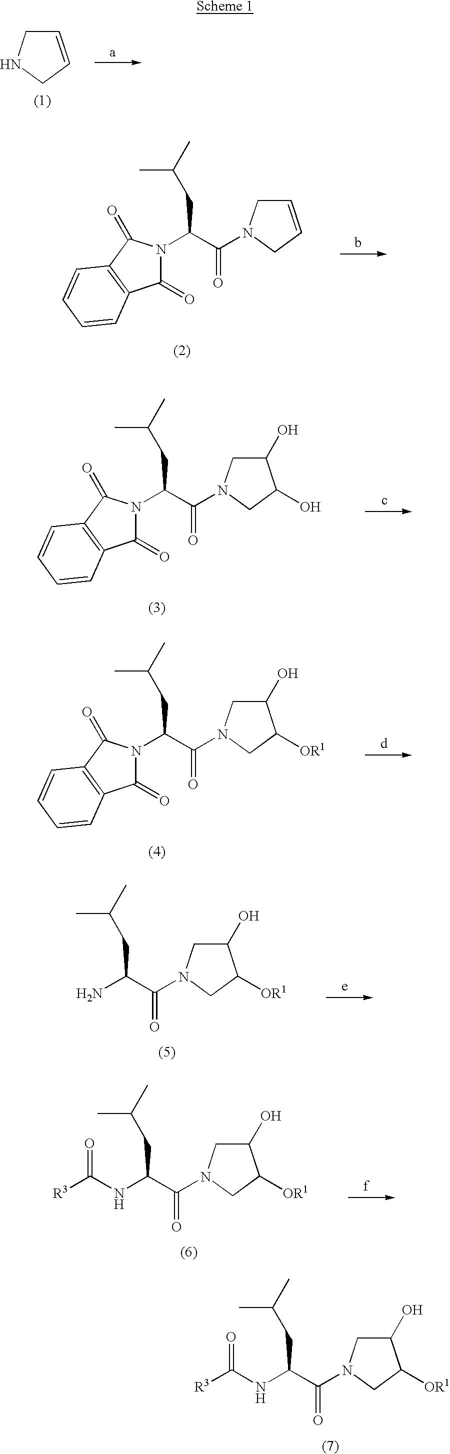

a) N-Phthaloyl-leuOH, N-methylmorpholine, EDC, HOBt, CH₂Cl₂; b) NMMO, OsO_{4;} c) NaH, RBr, DMF; d) H₂NNH₂, EtOH; e) Where R=Z: PhCH₂OCOCl, Et₃N, CH₂Cl₂, otherwise RCOOH, N-methylmorpholine, EDC, HOBt, CH₂Cl₂; f) Dess-Martin reagent, CH₂Cl₂

Compounds of the general formula (I), wherein X is O, are prepared by methods shown in Scheme 1. 3-Pyrroline 1-Scheme-1 is reacted with N-Phthaloyl-leuOH, in the presence of N-methylmorpholine, N-ethyl-N′(dimethylaminopropyl)-carbodiimide, and 1-hydroxybenzotriazole in dichloromethane (CH₂Cl₂) to give 2-Scheme-1. The compound 2 is then reacted with osmium tetroxide and N-methylmorpholine N-oxide to give 3-Scheme-1. This is subsequently treated with sodium hydride and R′-bromide in dimethyl formamide to give 4-Scheme-1. Compound 4 is then reacted with hydrazine hydrate in ethanol to afford the amine 5-Scheme-1. Where R³ is Z, compound 5 is reacted with a benzyl-chloroformate and triethylamine in dichloromethane. Where R³ is not 7, the compound 5 is reacted with an appropriate carboxylic acid in the presence of N-methylmorpholine, N-ethyl-N′(dimethylaminopropyl)-carbodiimide, and 1-hydroxybenzotriazole in dichloromethane to give compound 6-Scheme-1. Compound 6 is then treated with Dess-Martin reagent in dichloromethane to give the final product 7-Scheme-1.

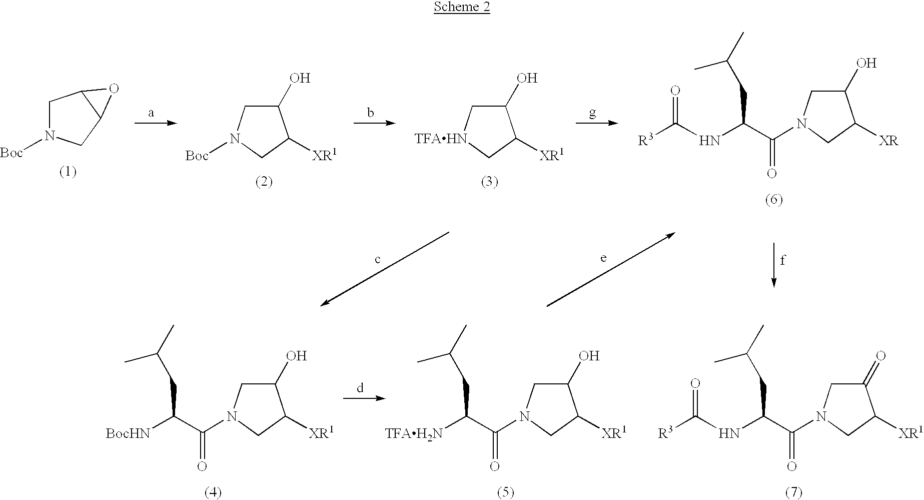

a) R¹XH/Na; b) TfA/CH₂Cl₂; c) N-Boc-leuOH, N-methylmorpholine, EDC, HOAt, CH₂Cl₂; d) TFA/CH₂Cl₂; e) R³COOH, N-methylmorpholine, EDC, HOAt, CH₂Cl₂; f) Dess-Martin reageant, CH₂Cl₂; g) for R³=Z: Z-leuOH, EDC, HOAt, CH₂Cl₂

Compounds of the general formula (I), wherein XR¹ is OCH₂Ph, SCH₂Ph, are prepared by the method shown in Scheme 2. The epoxide 1-scheme-2 is treated with the in situ generated sodium salt of the alcohol or thiol, R¹XH, in excess of the alcohol as solvent, or in an appropriate solvent such as methanol, for the thiol reagent, to afford 2-Scheme-2. This compound is deprotected using trifluoroacetic acid in dichloromethane to give 3-Scheme-2. Compound 3 is reacted with N-t-butyloxycarbonyl leucine in the presence of N-methylmorpholine, N-ethyl-N¹-butyloxycarbonyl leucine in the presence of N-methylmorpholine, N-ethyl-N¹-(dimethylaminopropyl)-carbodiimide and 1-hydroxy-7-azabenzotriazole in dichloromethane to give 4-Scheme-2. Compound 4 is then deprotected with trifluoroacetic acid in dichloromethane to give 5-Scheme -2. This compound is then treated with a carboxylic acid having the formula R³COOH, N-methylmorpholine, N-ethyl-N′(dimethylaminopropyl)-carbodiimide, and HOAt in dichloromethane to give 6-Scheme-2. Alternatively, 6-Scheme-2 can be produced directly from 2-Scheme-2, where R³ is Z, by treating 2-Scheme-2 with Z-leuOH, N-ethyl-N′(dimethylaminopropyl)-carbodiimide, and HOAt in dichloromethane. Compound 6 is then treated with Dess-Martin reagent in dichloromethane to give the final product 7-Scheme-2.

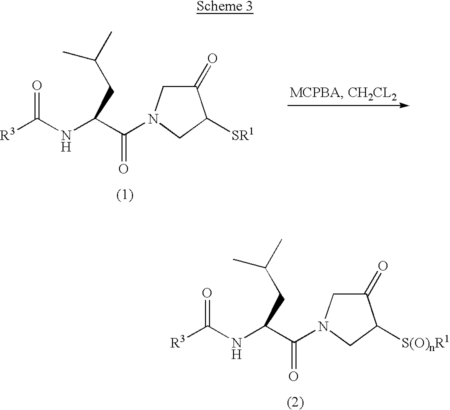

Compounds of the general formula (I), wherein X is S, SO, or SO₂ are prepared by the method shown in Scheme 3. According to this method, 1-Scheme-3 in dichloromethane is treated with m-chloroperoxybenzoic acid to give 2-Scheme-3.

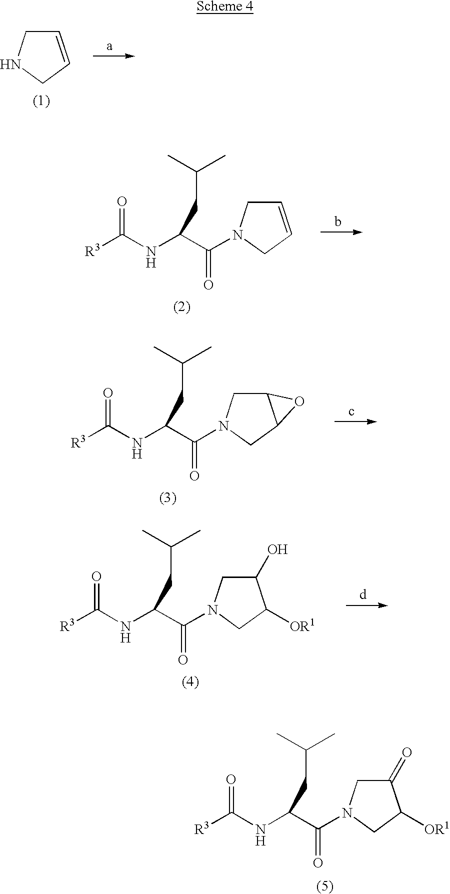

a) eg for R³=Z: Z-leuOH, N-methylmorpholine, EDC, HOBt, CH₂Cl₂; b) MCPBA, CH₂Cl₂; c) R¹OH, KO^{t}Bu, THF; d) Dess-Martin reagent, CH₂Cl₂.

Compounds of the general formula (I), wherein R¹ is a substituted phenyl group are prepared by the method shown in Scheme 4. Where R³ is Z, 3-pyrroline 1-Scheme4 is treated with Z-leuOH, N-methylmorpholine, N-ethyl-N′(dimethylaminopropyl)-carbodiimide, and HOBt (1-hydroxy benzotriazole) in dichloromethane to give 2-Scheme-4. This compound in dichloromethane is then treated with m-chloroperoxybenzoic acid to give the epoxide 3-Scheme4. Compound 3 is then reacted with the alcohol R²OH and potassium tert-butoxide in tetrahydrofuran to afford 4-Scheme4, which is subsequently treated with Dess-Martin reagent in dichloromethane to provide the final product 5-Scheme4

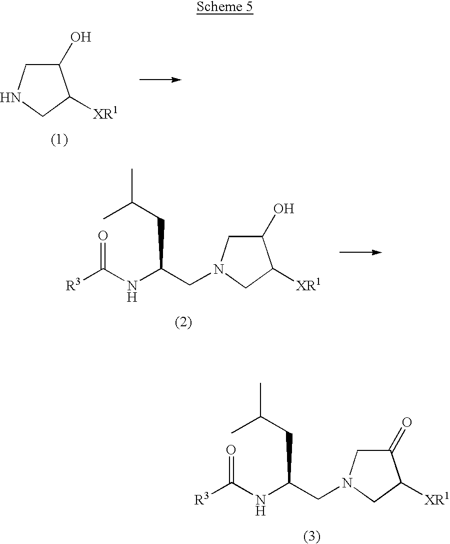

a) eg for R=Z: Z-leu-H. NaH(OAc)₃,Et₃N, CH₃OH; b) (COCl)_{2,} DMSO, Et₃N, CH₂Cl₂

Compounds having the general formula (I), wherein Y is H₂ are prepared according to the method of Scheme 5. In particular, where R³ is Z, 1-Scheme-5 is treated with Z-leucinal, sodium triacetoxyborohydride, and triethylamine in methanol to afford 2-Scheme-5. This compound is then is then oxidized using Swern conditions (oxalyl chloride, DMSO, triethyl-amine) to afford 2-Scheme-5.

The starting materials used herein are commercially available or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well-known to the art. The methods of peptide synthesis generally set forth by Bodansky et al., THE PRACTICE OF PEPTlDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, Ill., 1984, are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of formula (I) are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic acid. Certain of the compounds form inner salts or zwitterions which may be acceptable. Cationic salts are prepared by treating the parent compound with an excess of an alkaline reagent, such as a hydroxide, carbonate, or alkoxide, containing the appropriate cation; or with an appropriate organic amine. Cations such as Li⁺, Na⁺, K⁺, Ca⁺⁺, Mg⁺⁺ and NH₄⁺are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

This invention also provides a pharmaceutical composition which comprises a compound according to formula (l) and a pharmaceutically acceptable carrier, diluent or excipient. Accordingly, the compounds of formula (I) may be used in the manufacture of a medicament. Pharmaceutical compositions of the compounds of formula (I) prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water, or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, or sodium citrate.

Alternately, these compounds may be encapsulated, tableted, or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly or filled into a soft gelatin capsule.

For rectal administration, the compounds of this invention may also be combined with excipients such as cocoa butter, glycerin, gelatin or polyethylene glycols and molded into a suppository.

The compounds of formula (I) are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of cysteine proteases of the cathepsin family, most particularly as inhibitors of cathepsin K. The present invention also provides useful compositions and formulations of said compounds, including pharmaceutical compositions and formulations of said compounds.

The present compounds are useful for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Metastatic neoplastic cells also typically express high levels of proteolytic enzymes that degrade the surrounding matrix, and certain tumors and metastatic neoplasias may be effectively treated with the compounds of this invention.

The present invention also provides methods of treatment of diseases caused by pathological levels of proteases, particularly cysteine and serine proteases, more particularly cysteine proteases, even more particularly as inhibitors of cysteine proteases of the papain superfamily, yet more particularly cysteine proteases of the cathepsin family, which methods comprise administering to an animal, particularly a mammal, most particularly a human in need thereof a compound of the present invention. The present invention especially provides methods of treatment of diseases caused by pathological levels of cathepsin K, which methods comprise administering to an animal, particularly a mammal, most particularly a human in need thereof, an inhibitor of cathepsin K, including a compound of the present invention. The present invention particularly provides methods for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy, and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease, hypercalcemia of malignancy, and metabolic bone disease.

This invention further provides a method for treating osteoporosis or inhibiting bone loss which comprises internal administration to a patient of an effective amount of a compound of formula (I), alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

In accordance with this invention, an effective amount of the compounds of formula (I) is administered to inhibit the protease implicated with a particular condition or disease. Of course, this dosage amount will further be modified according to the type of administration of the compound. For example, “effective amount” for acute therapy, parenteral administration of a compound of formula (I) is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

Prodrugs of compounds of the present invention may be prepared by any suitable method. For those compounds in which the prodrug moiety is a ketone functionality, specifically ketals and/or hemiacetals, the conversion may be effected in accordance with conventional methods.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

The compounds of this invention may be tested in one of several biological assays to determine the concentration of a compound which is required to have a given pharmacological effect.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 μM final substrate concentration in the assays. All assays contained 10% DMSO. Independent experiments found that this level of DMSO had no effect on enzyme activity or kinetic constants. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):

v=VₘA/[Kₐ(1+I/K_{i,app})+A] (1)

where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:

[AMC]=vₛₛt+(v₀−vₛₛ)[1−exp(−k_{obs}t)]/k_{obs} (2)

where [AMC] is the concentration of product formed over time t, v₀ is the initial reaction velocity, and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).

One skilled in the art would consider any compound with a Kᵢ of less than 50 micromolar to be a potential lead compound. Preferably, the compounds used in the method of the present invention have a Kᵢ value of less than 1micromolar. Most preferably, said compounds have a Kᵢ value of less than 200 nanomolar.

Human Osteoclast Resorption Assay

Aliquots of osteoclastoma-derived cell suspensions were removed from liquid nitrogen storage, warmed rapidly at 37° C. and washed ×1 in RPMI-1640 medium by centrifugation (1000 rpm, 5 min at 4° C.). The medium was aspirated and replaced with murine anti-HLA-DR antibody, diluted 1:3 in RPMI-1640 medium, and incubated for 30 minutes on ice. The cell suspension was mixed frequently.

The cells were washed×2 with cold RPMI-1640 by centrifugation (1000 rpm, 5 min at 4° C.) and then transferred to a sterile 15 mL centrifuge tube. The number of mononuclear cells were enumerated in an improved Neubauer counting chamber.

Sufficient magnetic beads (5/mononuclear cell), coated with goat anti-mouse IgG, were removed from their stock bottle and placed into 5 mL of fresh medium (this washes away the toxic azide preservative). The medium was removed by immobilizing the beads on a magnet and is replaced with fresh medium.

The beads were mixed with the cells and the suspension was incubated for 30 minutes on ice. The suspension was mixed frequently. The bead-coated cells were immobilized on a magnet and the remaining cells (osteoclast-rich fraction) were decanted into a sterile 50 mL centrifuge tube. Fresh medium was added to the bead-coated cells to dislodge any trapped osteoclasts. This wash process was repeated×10. The bead-coated cells were discarded.

The osteoclasts were enumerated in a counting chamber, using a large-bore disposable plastic pasteur pipette to charge the chamber with the sample. The cells were pelleted by centrifugation and the density of osteoclasts adjusted to 1.5×10⁴/mL in EMEM medium, supplemented with 10% fetal calf serum and 1.7 g/liter of sodium bicarbonate. 3 mL aliquots of the cell suspension ( per treatment) were decanted into 15 mL centrifuge tubes. These cells were pelleted by centrifugation. To each tube 3 mL of the appropriate treatment was added (diluted to 50 μM in the EMEM medium). Also included were appropriate vehicle controls, a positive control (87MEM1 diluted to 100 ug/ml) and an isotype control (IgG2a diluted to 100 ug/mL). The tubes were incubated at 37° C. for 30 minutes.

0.5 mL aliquots of the cells were seeded onto sterile dentine slices in a 48-well plate and incubated at 37° C. for 2 hours. Each treatment was screened in quadruplicate. The slices were washed in six changes of warm PBS (10 mL/well in a 6-well plate) and then placed into fresh treatment or control and incubated at 37° C. for 48 hours. The slices were then washed in phosphate buffered saline and fixed in 2% glutaraldehyde (in 0.2M sodium cacodylate) for 5 minutes, following which they were washed in water and incubated in buffer for 5 minutes at 37° C. The slices were then washed in cold water and incubated in cold acetate buffer/fast red garnet for 5 minutes at 4° C. Excess buffer was aspirated, and the slices were air dried following a wash in water.

The TRAP positive osteoclasts were enumerated by bright-field microscopy and were then removed from the surface of the dentine by sonication. Pit volumes were determined using the Nikon/Lasertec ILM21W confocal microscope.

### EXAMPLES

In the following synthetic examples, unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

### Preparation of 3-Benzyloxy-1-(N-carbobenzyloxy-L-leucyl)pyrrolidin-4-one

(a) 3-Benzyloxy-1-tert-butoxycarbonyl-4-hydroxypyrrolidine

To a solution of sodium (0.12 g) in benzyl alcohol (3.69 g) was added 1-tert-butoxy-carbonyl-3,4-epoxypyrrolidine (1.07 g) and the mixture stirred at 60° C. for 15 hours. Water was added and the mixture extracted with dichloromethane. The organic extract was dried (magnesium sulphate) and evaporated down under reduced pressure. The residue was washed with cold hexane and dried under vacuum to give the title compound (0.88 g) as a white solid. ¹H NMR (CDCl₃) δ: 1.46 (s, 9H), 3.25-3.7 (m, 4H), 3.91 (s, 1H), 4.30 (s, 1H),4.54 (m, 2H), 4.70 (d, 1H), 7.3-7.45 (m, 5H).

(b) 3-Benzyloxy-4-hydroxypyrrolidine trifluoroacetate

A solution of 3-benzyloxy-1-tert-butoxycarbonyl-4-hydroxypyrrolidine (0.81 g) and trifluoroacetic acid (4 ml) in dichloromethane (16 ml) was stirred at room temperature for 15 hours. The solution was evaporated down under reduced pressure to give the title compound (1.31 g) as a tan-coloured liquid. ¹H NMR (D₂O) 67 : 3.15-3.5 (m, 4H), 4.09 (d, 1H), 4.41 (d, 1H), 4.50 (s, 2H), 7.28 (s, 5H).

(c) 3-Benzyloxy-4-hydroxy-1-(N-carbobenzyloxy-L-leucyl)pyrrolidine

A solution of 3-benzyloxy-4-hydroxypyrrolidine trifluoroacetate (0.29 g), N-methyl-morpholine (0.42 g), 1-(3-dimethylaminopropyl)-3-ethylcarbodiimide hydrochloride (0.16 g), 1-hydroxy-7-azabenzotriazole (0.10 g) and N-carbobenzyloxy-L-leucine (0.18 g) in dichloromethane (25 ml) was stirred at room temperature for 15 hours. Solvent was evaporated off under reduced pressure and the residue dissolved in ethyl acetate (25 ml). The solution was washed successively with 1N hydrochloric acid, saturated potassium carbonate solution, water and brine, dried (magnesium sulphate) and evaporated down under reduced pressure to give the title compound (0.28 g) as a pale tan oil. ¹H NMR (CDCl₃) δ: 0.9-1.05 (m, 6H), 1.4-1.6 (m, 3H), 3.35-4.05 (m, 6H), 4.25-4.7 (m, 4H), 5.08 (m, 2H), 5.51 (m, 1H), 7.35 (m, 10H).

(d) 3-Benzyloxy-1-(N-carbobenzyloxy-L-leucyl)pyrrolidin-4-one

A mixture of 3-benzyloxy-4-hydroxy-1-(N-carbobenzyloxy-L-leucyl)pyrrolidine (0.15 g) and Dess-Martin's reagent (0.31 g) in dichloromethane (10 ml) was stirred at room temperature for 2 hours. The solution was diluted with ether and treated with saturated sodium bicarbonate solution containing an excess of sodium thiosulphate. The ether layer was washed successively with saturated sodium bicarbonate solution and water, dried (magnesium sulphate) and evaporated down under reduced pressure to give the title compound (85 mg) as a colourless oil. ¹H NMR (CDCl₃) δ: 0.85-1.0 (m, 6H), 1.35-1.7 (m, 3H), 3.45-3.75 (m, 1H), 3.75-4.0 (m, 2H), 4.0-4.35 (m, 2H), 4.35-4.5 (m, 1H), 4.5-4.75 (m, 1H), 4.75-4.95 (m, 1H), 5.08 (s, 2H), 5.4 (m, 1H), 7.35 (m, 10H).

### Example 2

### Preparation of 3-Benzylthio-1-(N-carbobenzyloxy-L-leucyl)pyrrolidin-4one

(a) 3-Benzylthio-1-tert-butoxycarbonyl-4-hydroxypyrrolidine

A sodium methoxide solution (50 mg sodium in 5 ml methanol) was added to a solution of benzyl mercaptan (0.68 g) and 1-tert-butoxycarbonyl-3,4-epoxypyrrolidine (1.0 g) in dry methanol (5 ml) and the solution stirred at 50° C. for 24 hours. The solution was evaporated down under reduced pressure and water (20 ml) added. The mixture was extracted with dichloromethane (3×20 ml) and the combined extracts washed with water and brine, dried (magnesium sulphate) and evaporated down under reduced pressure to give the title compound (1.54 g) as a white solid, m.p. 97-8°. ¹H NMR (CDCl₃) δ: 1.45 (s, 9H), 3.02 (br.s, 1H), 3.2-3.4 (m, 2H), 3.7 (m, 2H), 3.80 (d, 2H), 4.15 (m, 1H), 7.30 (m, 5H).

(b) 3-Benzylthio-4-hydroxypyrrolidine hydrochloride

To a solution of 3-benzylthio-1-tert-butoxycarbonyl-4-hydroxypyrrolidine (0.93 g) in dry ethyl acetate (10 ml) at 0′ C. was introduced dry hydrogen chloride for 4 minutes and the solution stirred at 0° C. for 1 hour. Solvent was removed under reduced pressure to give the title compound (0.77 g) as a pale buff gum. ¹H NMR (DMSO-d₆) δ: 2.95-3.25 (m, 4H), 3.88 (d, 2H), 3.95 (d, 1H), 4.23 (m, 1H), 7.3 (m, 5H), 9.45 (br.s, 2H).

(c) 3-Benzylthio-4-hydroxy-1-(N-carbobenzyloxy-L-leucyl)pyrrolidine

In a manner similar to Example 1(c) reaction of 3-benzylthio-4-hydroxypyrrolidine hydrochloride (246 mg), N-methylmorpholine (506 mg), 1-(3-dimethylaminopropyl)-3-ethyl-carbodiimide hydrochloride (230 mg), 1-hydroxybenzotriazole (153 mg) and N-carbobenzyloxy-L-leucine (265 mg) in dichloromethane (20 ml) followed by chromatography over silica using 1:1 ethyl acetate:hexane gave the title compound (284mg) as a colourless gum. ¹ NMR (CDCl₃) δ: 0.95 (m, 6H), 1.3-1.8 (m, 3H), 2.95-3.2 (m, 1H), 3.25-3.55 (m, 2H), 3.55-3.9 (m, 4H), 3.9-4.3 (m, 2H), 4.45 (m, 1H), 5.05 (m, 2H), 5.45 (d, 1H), 7.33 (m, 10H).

(d) 3-Benzylthio-1-(N-carbobenzyloxy-L-leucyl)pyrrolidin-4-one

In a manner similar to Example 1(d) reaction of 3-benzylthio-4-hydroxy-1-(N-carbobenzyloxy-L-leucyl)pyrrolidine (0.58 g) and Dess-Martin's reagent (1.07 g) in dichloromethane (10 ml) followed by chromatography over silica using 4:1 hexane:ethyl acetate gave the title compound (0.40 g) as a pale yellow oil. ¹H NMR (DMSO-d₆) δ: 0.87 (m, 6H), 1.50 (m, 2H), 1.64 (m, 1H), 3.61 (m, 2H), 3.87 (m, 2H), 4.1 (m, 2H), 4.27 (m, 1H), 5.03 (s, 2H), 6.91 (d, 1H), 7.3 (m, 10H).

### Example 3

### Preparation of 3-Benzylsulfinyl-1-N-carbobenzyloxy-L-leucyl)pyrrolidin-4-one

To a solution of 3-benzylthio-1-N-carbobenzyloxy-L-leucyl)pyrrolidin-4-one (0.16 g) in dichloromethane (5 ml) at −60° was added m-chloroperoxybenzoic acid (77 mg) and the solution stirred at −60° for 1 hour. The solution was washed successively with 5% NaH₂SO₃ solution, saturated sodium bicarbonate solution and water, dried (magnesium sulphate) and evaporated down under reduced pressure to give the title compound (93 mg) as a glassy yellow solid. ¹H NMR (CDCl₃) δ: 0.95 (m, 6H), 1.4-1.8 (m, 3H), 3.8-4.7 (m, 8H), 5.05 (m, 2H), 5.3 (m, 1H), 7.3-7.45 (m, 10H).

### Example 4

Preparation of 3-Benzylsulfonyl-1-(N-carbobenzyloxy-L-leucyl)pyrrolidin-4-one

To a solution of 3-benzylthio-1-(N-carbobenzyloxy-L-leucyl)pyrrolidin-4-one (0.16 g) in dichloromethane (5 ml) at room temperature was added m-chloroperoxybenzoic acid (147 mg) and the solution stirred for 1 hour. The solution was washed successively with 5% NaH₂SO₃ solution, saturated sodium bicarbonate solution and water, dried (magnesium sulphate) and evaporated down under reduced pressure to give the title compound (0.13 g) as a glassy yellow solid. ¹H NMR (CDCl₃) δ: 0.95 (m, 6H), 1.4-1.85 (m, 3H), 3.9-4.25 (m, 3H), 4.25-4.6 (m, 4H), 4.6-4.8 (m, 1H), 5.05 (m, 2H), 5.3 (m, 1H), 7.3-7.55 (m, 10H).

### Example 5

### Preparation of 1-[2-(Benzyloxycarbonylamino)-4-methylpentyl]-3-benzylthiopyrrolidin-4-one

(a) 1-[2-(Benzyloxycarbonylamino)-4-methylpentyl]-3-benzylthio-4-hydroxypyrrolidine

To a stirring solution of 3-benzylthio-4-hydroxypyrrolidine hydrochloride (0.25 g) in dichloromethane (10 ml) was added triethylamine (0.12 g) and a solution of N-carbobenzyl-oxy-L-leucinaldehyde (0.30 g) in methanol (10 ml). The solution was stirred for 30 minutes, sodium triacetoxyborohydride (0.53 g) added and the mixture stirred for 18 hours. Water (10 ml) was added and the mixture stirred for 15 minutes. The organic layer was separated, washed successively with saturated sodium bicarbonate solution and brine, dried (magnesium sulphate) and evaporated down under reduced pressure. Chromatography over silica using 1:1 ethyl acetate:hexane gave the title compound (0.31 g) as a colourless oil. ¹H NMR (CDCl₃) δ: 0.95 (m, 6H), 1.30 (m, 2H), 1.65 (m, 1H), 2.05 (m, 1H), 2.35-2.6 (m, 3H), 2.70 (m, 1H),2.95 (m, 1H), 3.15-3.3 (m, 1H), 3.78 (m, 3H), 4 03 (m, 1H), 4.59 (m, 1H), 5.11 (m, 2H), 7.33 (m, 10H).

(b) 1-[2-(Benzyloxycarbonylamino)-4-methylpentyl]-3-benzylthiopyrrolidin-4-one

To a stirring solution of oxalyl chloride (90 mg) in dichloromethane (3 ml) at −65 ° was slowly added a solution of dimethyl sulphoxide (0.12 g) in dichloromethane (3 ml), the mixture stirred for 5 minutes, then a solution of 1-[2-(benzyloxycarbonylamino)-4-methyl-pentyl]-3-benzylthio-4-hydroxypyrrolidine (285 mg) in dichloromethane (4 ml) slowly added. The mixture was stirred at −65° for 20 minutes, triethylamine (325 mg) slowly added and stirring continued for 18 hours at room temperature. Water (5 ml) was added, the mixture stirred for 10 minutes and the aqueous layer extracted with dichloromethane (2×5 ml). The combined dichloromethane layers were dried (magnesium sulphate) and evaporated down under reduced pressure. Chromatography over silica using 4:1 hexane:ethyl acetate gave the title compound (90 mg) as a tan gum. ¹H NMR (CDCl₃) δ: 0.90 (d, 6H), 1.33 (m, 2H), 1.67 (m, 1H), 2.50 (m, 2H), 2.6-2.8 (m, 2H), 2.95-3.3 (m, 3H), 3.78 (d, 2H), 3.95 (d, 1H), 4.6 (m, 1H), 5.10, (s, 2H), 7.31 (m, 10H).

### Example 6

### Preparation of 3-Benzylthio-1-[N-(2-quinolinecarbonyl)-L-leucyl]pyrrolidin-4-one

(a) 3-Benzylthio-4-hydroxy-1-[N-(tert-butoxycarbonyl)-L-leucyl]pyrrolidine

In a manner similar to Example 1(c) reaction of 3-benzylthio-4-hydroxypyrrolidine trifluoroacetate (1.42 g), N-methylmorpholine (2.3 g), 1-(3-dimethylaminopropyl)-3-ethyl-carbodiimide hydrochloride (1.06 g), 1-hydroxybenzotriazole (0.71 g) and N-(tert-butoxycarbonyl)-L-leucine (1.11 g) in dichloromethane (50 ml) followed by chromatography over silica using 1:1 ethyl acetate:hexane gave the title compound (1.10 g) as a colourless oil. ¹H NMR (CDCl₃) δ: 0.95 (m, 6H), 1.42 (d, 9H), 1.64 (m, 3H), 2.95-3.2 (m, 1H), 3.3-3.55 (m, 2H), 3.8 (m, 4H), 3.95-4.25 (m, 2H), 4.35 (m, 1H), 5.15 (m, 1H), 7.3 (m, 5H).

(b) 3-Benzylthio-4-hydroxy-1-L-leucylpyrrolidine trifluoroacetate

In a manner similar to Example 1(b) reaction of 3-benzylthio-4-hydroxy-4-hydroxy-1-[N-(tert-butoxycarbonyl)-L-leucyl]pyrrolidine (0.92 g) and trifluoroacetic acid (2 ml) in dichloromethane (8 ml) gave the title compound (1.35 g) as a tan-coloured oil.

(c) 3-Benzylthio-4-hydroxy-1-[N-(2-quinolinecarbonyl)-L-leucyl]pyrrolidine

In a manner similar to Example 1(c) reaction of 3-benzylthio-4-hydroxy-1-L-leucyl-pyrrolidine trifluoroacetate (0.70 g), N-methylmorpholine (0.83 g), 1-(3-dimethylamino-propyl)-3-ethylcarbodiimide hydrochloride (0.37 g), 1-hydroxybenzotriazole (0.25 g) and 2-quinolinecarboxylic acid (0.29 g) in dichloromethane (15 ml) followed by chromatography over silica using 1:1 ethyl acetate:hexane gave the title compound (0.27 g) as a colourless oil. ¹H NMR (CDCl₃) δ: 0.95 (m, 6H), 1.65-1.85 (m, 3H), 3.0-3.25 (m, 1H), 3.4-3.6 (m, 2), 3.7-4.05 (m, 4H), 4.15-4.3 (m, 2H), 4.95 (m, 1H), 7.32 (m, 5H), 7.61 (t, 1H), 7.75 (t, 1H), 7.85 (d, 1H), 8.15 (t, 1H), 8.35 (m, 2H), 8.80 (t, 1H).

(d) 3-Benzylthio-1-[N-(2-quinolinecarbonyl)-L-leucyl]pyrrolidin-4-one

In a manner similar to Example 1(d) reaction of 3-benzylthio-4-hydroxy-1-[N-(2-quinolinecarbonyl)-L-leucyl]pyrrolidine (0.25 g) and Dess-Martin's reagent (0.46 g) in dichloromethane (15 ml) followed by chromatography over silica using 1:1 hexane:ethyl acetate gave the title compound (0.12 g) as a colourless oil. ¹H NMR (CDCl₃) δ: 1.0 (m, 6H), 1.75 (m, 3H), 3.2-3.4 (m, 1H), 3.7-4.05 (m, 4H), 4.25-4.45 (m, 2H), 4.85-5.15 (m, 1H), 7.35 (m, 5H), 7.62 (t, 1H), 7.77 (t, 1H), 7.85 (d, 1H), 8.15-8.35 (m, 3H), 8.75 (m, 1H).

### Example 7

### Preparation of 3-tert-Butoxycarbonylmethoxy-1-(N-carbobenzyloxy-L-leucyl)pyrrolidin-4-one

(a) 1-(N-Phthaloyl-L-leucyl)-3-pyrroline

In a manner similar to Example 1(c) reaction of 3-pyrroline (2.23 g), N-methylmorpholine (16.6 g), 1-(3-dimethylaminopropyl)-3-ethylcarbodiimide hydrochloride (7.45 g), 1-hydroxybenzotriazole (5.99 g) and N-phthaloyl-L-leucine (8.62 g) in dichloromethane (100 ml) gave the title compound (9.75 g) as a white solid. ¹H NMR (CDCl₃) δ: 0.95 (m, 6H), 1.55 (m, 1H), 1.7-1,85 (m, 1H), 2.62 (m, 1H), 4.28 (m, 4H), 5.00 (d of d, 1H), 5.82 (d of d, 2H), 7.75 (m, 2H), 7.85 (m, 2H).

(b) 3,4-Dihydroxy-1-(N-phthaloyl-L-leucyl)pyrrolidine

To a solution of N-methylmorpholine-N-oxide (4.41 g) and osmium tetroxide (80 mg) in water (50 ml), acetone (20 ml) and tert-butanol (10 ml) was added 1-(N-phthaloyl-L-leucyl)-3-pyrroline (9.24 g) and the solution stirred at room temperature for 3 days. Sodium metabisulphite (2.3 g), magnesium silicate (fluorisil, 2.3 g) and sodium sulphate (4.6 g) were added and the mixture stirred for 30 minutes. Solid was filtered through Celite and washed with acetone. The filtrate was evaporated down under reduced pressure and dichloromethane (100 ml) was added. The mixture was washed successively with sodium thiosulphate solution, 1N hydrochloric acid, saturated sodium bicarbonate solution and brine, dried (magnesium sulphate) and evaporated down under reduced pressure to give the title compound (8.87 g) as a white solid. ¹H NMR (CDCl₃) δ: 0.95 (m, 6H), 1.55 (m, 1H), 1.75 (m, 1H), 2.35-2.65 (m, 1H), 3.1 (m, 2H), 3.3-3.6 (m, 2H), 3.7 (m, 2H), 4.25 (d, 2H), 4.95 (m, 1H), 7.75 (m, 2H), 7.9 (m, 2H).

(c) 3-tert-Butoxycarbonylmethoxy-4-hydroxy-1-(N-phthaloyl-L-leucyl)pyrrolidine

To a solution of 3,4-dihydroxy-1-(N-phthaloyl-L-leucyl)pyrrolidine (0.80 g) in dry dimethylformamide (10 ml) at −5° was added 60% sodium hydride in oil (0.10 g) and the mixture stirred for 30 minutes at −5°. Tert-butyl bromoacetate (568 mg) was added and the solution stirred for 16 hours at room temperature. The bulk of the solvent was removed under reduced pressure and dichloromethane (50 ml) and ice-water (50 ml) added. The aqueous layer was washed with dichloromethane (20 ml) and the combined organic layers washed with water, dried (magnesium sulphate) and evaporated down under reduced pressure. Chromatography over silica using 3:1 ethyl acetate:hexane gave the title compound (458 mg) as a colourless oil. ¹H NMR (CDCl₃) δ: 0.95 (m, 6H), 1.5 (m, 9H), 1.75 (m, 2H), 2.3-2.75 (m, 1H), 3.35 (m, 1H), 3.5-3.8 (m, 3H), 3.8-4.0 (m, 2H), 4.1-4.3 (m, 2H), 5.0 (m, 2H), 7.75 (m, 2H), 7.85 (m, 2H).

(d) 3-tert-Butoxycarbonylmethoxy4-hydroxy-1-L-leucylpyrrolidine

A solution of 3-tert-butoxycarbonylmethoxy-4-hydroxy-1-(N-phthaloyl-L-leucyl)pyrrolidine (440 mg) and hydrazine hydrate (0.26 g) in ethanol (10 ml) was stirred under reflux for 1 hour. The white solid that formed was filtered off, washed with ethanol and dichloromethane and the filtrate evaporated down under reduced pressure. Water (5 ml) and dichloromethane (5 ml) were added, the aqueous layer washed with dichloromethane (5 ml) and the combined organic layers washed with water, dried (magnesium sulphate) and evaporated down under reduced pressure to give the title compound (202 mg) as a colourless oil. ¹H NMR (CDCl₃) δ: 0.95 (m, 6H), 1.4 (m, 1H), 1.50 (s, 9H), 1.85 (m, 2H), 3.4-3.85 (m, 7H), 3.95 (m, 2H), 4.3 (m, 2H).

(e) 3-tert-Butoxycarbonylmethoxy-4-hydroxy-1-(N-carbobenzyloxy-L-leucyl)pyrrolidine

To a solution of 3-tert-butoxycarbonylmethoxy-4-hydroxy-1-L-leucylpyrrolidine (96 mg) and triethylamine (73 mg) in dichloromethane (2.5 ml) at 0′ was slowly added a solution of benzyl chloroformate (60 mg) in dichloromethane (2.5 ml) and the solution stirred at 4° for 3 hours. The solution was was washed with water, dried (magnesium sulphate) and evaporated down under reduced pressure to give the title compound (129 mg) as a colourless gum. ¹H NMR (CDCl₃) δ: 0.95 (m, 6H), 1.4 (m, 1H), 1.50 (s, 9H), 1.75 (m, 2H), 3.45-3.85 (m, 3H), 3.85-4.15 (m, 3H), 4.15-4.4 (m, 2H), 4.5 (m, 1H), 4.71 (d, 1H), 5.09 (d, 2H), 5.45 (m, 1H), 7.35 (m, 5H).

(f) 3-tert-Butoxycarbonylmethoxy-1-(N-carbobenzyloxy-L-leucyl)pyrrolidin-4-one

In a manner similar to Example 1(d) reaction of 3-tert-butoxycarbonylmethoxy-4-hydroxy-1-(N-carbobenzyloxy-L-leucyl)pyrrolidine (119 mg) and Dess-Martin's reagent (500 mg) in dichloromethane (5 ml) followed by chromatography over silica using 2:1 hexane:ethyl acetate gave the title compound (71 mg)as a pale yellow oil. ¹H NMR (CDCl₃) δ: 0.95 (m, 6H), 1.4 (m, 1H), 1.48 (d, 9H), 1.7 (m, 2H), 3.55-4.05 (m, 3H), 4.05-4.3 (m, 3H), 4.3-4.7 (m, 2H), 5.09 (s, 2H), 5.35 (m, 1H), 7.34 (s, 5H).

### Example 8

### Preparation of 3-(3-Methoxybenzyloxy)-1-(N-carbobenzyloxy-L-leucyl)pyrrolidin-4-one

(a) 3-Hydroxy-4-(3-methoxybenzyloxy)-1-(N-phthaloyl-L-leucyl)pyrrolidine

In a manner similar to Example 7(c) reaction of 3,4-dihydroxy-1-(N-phthaloyl-L-leucyl)-pyrrolidine (0.83 g), 60% sodium hydride in oil (0.12 g) and 1-bromomethyl-3-methoxy-benzene (0.72 g) in dry dimethylformamide (50 ml) followed by chromatography over silica using 4:1 ethyl acetate:hexane gave the title compound (0.29 g) as a colourless oil. ¹H NMR (CDCl₃) δ: 0.95 (m, 6H), 1,55 (m, 1H), 1.65 (m, 1H), 2.35-2.75 (m, 2H), 3.45-3.75 (m, 4H), 3.81 (m, 3H), 4.0 (m, 1H), 4.25 (m, 1H), 4.4-4.7 (m, 2H), 4.95 (m, 1H), 6.85 (m, 3H), 7.3 (m, 1H), 7.35 (m, 2H), 7 45 (m, 2H).

(b) 3-Hydroxy-4-(3-methoxybenzyloxy)-1-L-leucylpyrrolidine

In a manner similar to Example 7(d) reaction of 3 hydroxy-4 -(3-methoxybenzyloxy)-1-(N-phthaloyl-L-leucyl)pyrrolidine (0.26 g) and hydrazine hydrate (0.15 g) in ethanol (10 ml) gave the title compound (0.11 g) as a colourless oil. ¹H NMR (CDCl₃) δ: 0.93 (m, 6H), 1.4 (m, 2H), 1.85 (m, 1H), 3.35-3.8 (m, 6H), 3.82 (s, 3H), 4.05 (m, 1H), 4.3 (m, 1H), 4.5-4.7 (m, 2H), 6.9 (m, 4H), 7.3 (m, 2H).

(c) 3-Hydroxy-4-(3-methoxybenzyloxy)-1-(N-carbobenzyloxy-L-leucyl)pyrrolidine

In a manner similar to Example 7(e) reaction of 3-hydroxy-4-(3-methoxybenzyloxy)-1-L-leucyl)pyrrolidine (90 mg), triethylamine (73 mg) and benzyl chloroformate (60 mg) in dichloromethane (5 ml) gave the title compound (123 mg) as a colourless gum. ¹H NMR (CDCl₃) δ: 0.95 (m, 6H), 1.3-1.55 (m, 2H), 1.7 (m, 1H), 2.5-2.8 (m, 1H), 3.4-3.95 (m, 3H), 3.82 (s, 3H), 4.05 (m, 1H), 4.3 (m, 1H), 4.4-4.55 (m, 1H), 4.60 (s, 2H), 4.7 (m, 1H), 5.07 (s, 2H), 5.4 (m, 1H), 6.9 (m, 3H), 7.35 (m, 6H).

(d) 3-(3-Methoxybenzyloxy)-1(N-carbobenzyloxy-L-leucyl)pyrrolidin-4-one

In a manner similar to Example 1(d) reaction of 3-hydroxy-4-(3-methoxybenzyloxy)-1-(N-carbobenzyloxy-L-leucyl)pyrrolidine (113 mg) and Dess-Martin's reagent (500 mg) in dichloromethane (5 ml) followed by chromatography over silica using 2:1 hexane:ethyl acetate gave the title compound (50 mg) as a colourless oil. ¹H NMR (CDCl₃) δ: 0.95 (m, 6H), 1.3-1.8 (m, 3H), 3.45-3.65 (m, 1H), 3.81 (s, 3H), 3.85-4.0 (m, 1H), 4.0-4.25 (m, 2H),4.25-4.45 (m, 1H), 4.5-4.75 (m, 2H), 4.85 (m, 1H), 5.08 (m, 2H), 5.4 (m, 1H), 6.9 (m, 3H), 7.33 (m, 6H).

### Example 9

### Preparation of 3-(3-Methoxycarbonylbenzyloxy)-]-(N-carbobenzyloxy-L-leucyl)pyrrolidin-4-one

(a) 3-Hydroxy-4-(3-methoxycarbonylbenzyloxy)-1-(N-phthaloyl-L-leucyl)pyrrolidine

In a manner similar to Example 7(c) reaction of 3,4-dihydroxy-1-(N-phthaloyl-L-leucyl)-pyrrolidine (1.40 g), 60% sodium hydride in oil (0.18 g) and methyl-3-bromomethylbenzoate (1.08 g) in dry dimethylformamide (100 ml) followed by chromatography over silica using 3:1 ethyl acetate:hexane gave the title compound (0.92 g) as a colourless oil. ¹H NMR (CDCl₃) δ: 0.95 (m, 6H), 1.45-1.8 (m, 3H), 3.3-3.8 (m, 3H), 3.95 (m, 3H), 4.05 (m, 1H), 4.3 (m, 1H), 4.5-4.75 (m, 2H), 4.95 (m, 1H), 7.5 (m, 2H), 7.75 (m, 2H), 7.85 (m, 2H), 8.0 (m, 2H).

(b) 3-Hydroxy-4-(3-methoxycarbonylbenzyloxy)-1-L-leucyl-pyrrolidine

In a manner similar to Example 7(d) reaction of 3-hydroxy-4-(3-methoxycarbonyl-benzyloxy)-1-(N-phthaloyl-L-leucyl)pyrrolidine (0.91 g) and hydrazine hydrate (0.45 g) in ethanol (15 ml) gave the title compound (0.64 g) as a pale buff gum. ¹H NMR (CDCl₃) δ: 0.95 (m, 6H), 1.35 (m, 2H), 1.8 (m, 1H), 3.4-3.8 (m, 5H), 3.94 (s, 3H), 4.05 (m, 1H), 4.35 (m, 1H), 4.55-4.75 (m, 2H), 7.5 (m, 2H), 8.0 (d, 2H).

(c) 3-Hydroxy-4-(3-methoxycarbonylbenzyloxy)-1-(N-carbobenzyloxy-L-leucyl)pyrrolidine

In a manner similar to Example 7(e) reaction of 3-hydroxy-4-(3-methoxycarbonyl-benzyloxy)-1L-leucyl-pyrrolidine (315 mg), triethylamine (218 mg) and benzyl chloroformate (179 mg) in dichloromethane (15 ml) gave the title compound (429 mg) as a colourless gum. ¹H NMR (CDCl₃) δ: 0.95 (m, 6H), 1.35-1.6 (m, 3H), 2.6 (m, 1H), 3.4-3.85 (m, 3H), 3.94 (s, 3H), 4.05 (m, 1H), 4.3 (m, 1H), 4.45 (m, 1H), 4.6 (m, 1H), 4.7 (d, 2H), 5.07 (s, 2H), 5.45 (m, 1H), 7.3 (m, 5H), 7.5 (m, 2H), 8.01 (m, 2H).

(d) 3-(3-Methoxycarbonylbenzyloxy)-1-N-carbobenzyloxy-L-leucyl)pyrrolidin-4-one

In a manner similar to Example 1(d) reaction of 3-hydroxy-4-(3-methoxycarbonyl-benzyloxy)-1-(N-carbobenzyloxy-L-leucyl)pyrrolidine (419 mg) and Dess-Martin's reagent (1.5 g) in dichloromethane (15 ml) followed by chromatography over silica using 2:1 hexane:ethyl acetate gave the title compound (176 mg) as a colourless oil. ¹H NMR (CDCl₃) δ: 0.95 (m, 6H), 1.35-1.6 (m, 3H), 3.35-3.6 (m, 1H), 3.96 (s, 3H), 4.0-4.3 (m, 2H), 4.3-4.5 (m, 2H), 4.6 (m, 1H), 4.7 (d, 1H), 4.85-5.0 (m, 1H), 5.08 (s, 2H), 5.4 (m, 1H), 7.34 (s, 5H), 7.45 (m, 1H), 7.55 (m, 1H), 8.05 (m, 2H).

### Example 10

### Preparation of 3-tert-Butoxycarbonylmethoxy-1-[N-(2-quinolinecarbonyl)-L-leucyl]pyrrolidin-4-one

(a) 3-tert-Butoxycarbonylmethoxy-4-hydroxy-1-[N-(2-quinolinecarbonyl)-L-leucyl]pyrrolidine-4-one

In a manner similar to Example 1(c) reaction of 3-tert-butoxycarbonylmethoxy-4-hydroxy-1-L-leucylpyrrolidine (96 mg), N-methylmorpholine (120 mg), 1-(3-dimethylamino-propyl)-3-ethylcarbodiimide hydrochloride (67 mg), 1-hydroxybenzotriazole (45 mg) and 2-quinolinecarboxylic acid (51 mg) in dichloromethane (5 ml) followed by chromatography over silica using 1:1 ethyl acetate:hexane gave the title compound (115 mg) as a colourless gum. ¹H NMR (CDCl₃) δ: 1.0 (m, 6H), 1.5 (m, 9H), 1.75 (m, 3H), 3.5-3.8 (m, 2H), 3.85-4.35 (m, 7H), 5.0 (m, 1H), 7.6 (t, 1H), 7.75 (t, 1H), 7.85 (d, 1H), 8.15 (d, 1H), 8.25 (m, 2 H), 8.75 (m, 1H).

(b) 3-tert-Butoxycarbonylmethoxy-1-[N-(2-quinolinecarbonyl)-L-leucyl]pyrrolidine4-one

In a manner similar to Example 1 (d) reaction of 3-tert-butoxycarbonylmethoxy-4-hydroxy-1-[N-(2-quinolinecarbonyl)-L-leucyl]pyrrolidine (105 mg) and Dess-Martin's reagent (650 mg) in dichloromethane (5 ml) followed by chromatography over silica using 2:1 hexane:ethyl acetate gave the title compound (87 mg) as a colourless gum. ¹H NMR (CDCl₃) δ: 1.05 (m, 6H), 1.5 (m, 9H), 1.8 (m, 3H), 3.55-3.8 (m, 1H), 3.8-4.0 (m, 1H), 4.0-4.5 (m, 4H), 4.5-4.75 (m, 1H), 4.85-5.25 (m, 1H), 7.62 (t, 1H), 7.79 (t, 1H), 7.87 (d, 1H), 8.15 (d, 1H), 8.25 (m, 2H), 8.75 (m, 1H).

### Example 11

Preparation of 1-[N-(2-Quinolinecarbonyl)-L-leucyl]-3-oxo-4-pyrrolidineoxyacetic acid

A solution of 3-tert-butoxycarbonylmethoxy-1-[N-(2-quinolinecarbonyl)-L-leucyl]pyrrolidin-4one (14 mg) and trifluoroacetic acid (0.1 ml) in dichloromethane (0.5 ml) was stirred at room temperature for 3 hours. Solvent was removed under reduced pressure and traces azeotroped with toluene to give the title compound (12 mg) as a buff foam. ¹H NMR (CDCl₃) δ: 0.95 (m, 6H), 1.5-1.9 (m, 3H), 3.5-4.5 (m, 6H), 4.5-4.7 (m, 1H),4.75-5.15 (m, 1H).

### Example 12

### Preparation of 3-(3-Methoxybenzyloxy)-1-[N-(2-quinolinecarbonyl)-L-leucyl]pyrrolidin-4-one

(a) 3-Hydroxy-4-(3-methoxybenzyloxy)-1-[N-(2-quinolinecarbonyl)-L-leucyl]pyrrolidine

In a manner similar to Example 1(c) reaction of 3-hydroxy-4-(3-methoxybenzyloxy)-1-L-leucylpyrrolidine (404 mg), N-methylmorpholine (488 mg), 1-(3-dimethylaminopropyl)-3-ethylcarbodiimide hydrochloride (275 mg), 1-hydroxybenzotriazole (184 mg) and 2-quinolinecarboxylic acid (208 mg) in dichloromethane (20 ml) followed by chromatography over silica using 3:1 ethyl acetate:hexane gave the title compound (519 mg) as a colourless gum. H NMR (CDCl₃) δ: 1.0 (m, 6H), 1.75 (m, 3H), 3.6 (m, 2H), 3.85 (m, 4H), 4.0-4.2 (m, 2H), 4.3 (m, 1H), 4.45-4.7 (m, 2H), 5.0 (m, 1H), 6.9 (m, 3H), 7.3 (m, 1H), 7.61 (t, 1H), 7.76 (t, 1H), 7.86 (d, 1H), 8.15 (d, 1H), 8.25 (m, 2H), 8.75 (m, 1H).

(b) 3-(3-Methoxybenzyloxy)-1-[N-(2-quinolinecarbonyl)-L-leucyl-3pyrrolidin-4-one

In a manner similar to Example 1 (d) reaction of 3-hydroxy-4-(3-methoxybenzyloxy)-1-[N-(2-quinolinecarbonyl)-L-leucyl]pyrrolidine (504 mg) and Dess-Martin's reagent (1.65 g) in dichloromethane (18 ml) followed by chromatography over silica using 2:1 hexane:ethyl acetate gave the title compound (448 mg) as a colourless oil. ¹H NMR (CDCl₃) δ: 1.0 (m, 6H), 1.75 (m, 3H), 3.45-3.75 (m, 1H), 3.85 (m, 3H), 3.9-4.45 (m, 3H), 4.5-4.75 (m, 2H), 4.75-5.2 (m, 2H), 6.95 (m, 3H), 7.3 (m, 1H), 7.62 (t, 1H), 7.78 (t, 1H), 7.86 (t, 1H), 8.14 (d, 1H), 8.3 (m, 2H), 8.7 (m, 1H).

### Example 13

### Preparation of 3-(4-Methoxybenzyloxy)-1-[N-(2-quinolinecarbonyl)-L-leucyl]pyrrolidin-4-one

(a) 3-Hydroxy-4-(4-methoxybenzyloxy)-1-(N-phthaloyl-L-leucyl)pyrrolidine

In a manner similar to Example 7(c) reaction of 3,4-dihydroxy-1-(N-phthaloyl-L-leucyl)-pyrrolidine (0.80 g), 60% sodium hydride in oil (0.10 g) and 1-bromomethyl-4-methoxy-benzene (0.58 g) in dry dimethylformamide (10 ml) followed by chromatography over silica using 3:1 ethyl acetate:hexane gave the title compound (322 mg) as a colourless oil. ¹H NMR (CDCl₃) δ:0.95 (m, 6H), 1.55 (m, 2H), 1.75 (m, 1H), 2.35-2.7 (m, 2H), 3.45-3.75 (m, 3H), 3.8 (m, 3H), 4.0 (m, 1H), 4.25 (m, 1H), 4.35-4.6 (m, 2H), 4.95 (m, 1H),6.9 (m, 2H), 7.25 (m, 2H), 7.7 (m, 2H), 7.85 (m, 2H).

(b) 3-Hydroxy-4-(4-methoxybenzyloxy)-1-L-leucylpyrrolidine

In a manner similar to Example 7(d) reaction of 3 hydroxy-4-(4-methoxybenzyloxy)-1-(N-phthaloyl-L-leucyl)pyrrolidine (0.31 g) and hydrazine hydrate (0.16 g) in ethanol (6 ml) gave the title compound (213 mg) as a colourless oil. ¹H NMR (CDCl₃) 67 : 0.95 (m, 6H), 1.4 (m, 2H), 1.8 (m, 1H), 3.3-3.8 (m, 5H), 3.81 (s, 3H), 4.0 (m, 1H), 4.25 (m, 1H), 4.4-4.65 (m, 2H), 6.9 (m, 2H), 7.3 (m, 2H).

(c) 3-Hydroxy-4-(4-methoxybenzyloxy)-1-[N-(2-quinolinecarbonyl)-L-leucyl]pyrrolidine

In a manner similar to Example 1(c) reaction of 3-hydroxy-4-(4-methoxybenzyloxy)-1-L-leucyl]pyrrolidine (203 mg), N-methylmorpholine (248 mg), 1-(3-dimethylaminopropyl)-3-ethylcarbodiimide hydrochloride (140 mg), 1-hydroxybenzotriazole (93 mg) and 2-quinolinecarboxylic acid (105 mg) in dichloromethane (10 ml) followed by chromatography over silica using 3:1 ethyl acetate:hexane gave the title compound (273 mg) as a colourless gum. ¹H NMR (CDCl₃) 67 : 1.0 (m, 6H), 1.5-1.9 (m, 3H), 3.5-3.7 (m, 2H), 3.8 (m, 3H), 3.95-4.2 (m, 2H), 4.3 (m, 1H), 4.5 (m, 1H), 4.6 (m, 2H), 5.0 (m, 1H), 6.9 (m, 2H), 7.3 (m, 2H), 7.61 (t, 1H), 7.76 (t, 1H), 7.86 (d, 1H), 8.15 (d, 1H), 8.25 (m, 2H), 8.75 (m, 1H).

(d) 3-(4Methoxybenzyloxy)-1-[N-(2-quinolinecarbonyl)-L-leucyl]pyrrolidin-1-one

In a manner similar to Example 1(d) reaction of 3-hydroxy-4-(4-methoxybenzyloxy)-1-[N-(2-quinolinecarbonyl)-L-leucyl]pytrolidine (262 mg) and Dess-Martin's reagent (1.20 g) in dichloromethane (10 ml) followed by chromatography over silica using 2:1 hexane:ethyl acetate gave 138 mg of the title compound as a colourless oil. ¹H NMR (CDCl₃) δ: 1.0 (m, 6H), 1.6-1.9 (m, 3H), 3.45-3.75 (m, 1H), 3.81 (m, 3H), 4.0-4.25 (m, 3H), 4.5-4.7 (m, 2H),4.7-5.0 (m, 1H), 5.1 (m, 1H), 6.9 (m, 2H), 7.3 (m, 2H), 7.62 (t, 1H), 7.78 (t, 1H), 7.88 (d, 1H), 8.15 (d, 1H), 8.3 (m, 2H), 8.7 (m, 1H).

### Example 14

### Preparation of 3-(3-Methoxycarbonylbenzyloxy)-1-[N-(2-quinolinecarbonyl)-L-leucyl]pyrrolidin-4-one

(a) 3-Hydroxy-4-(3-methoxycarbonylbenzyloxy)l-1-[N-(2-quinolinecarbonyl)-L-leucyl]pyrrolidine

In a manner similar to Example 1(c) reaction of 3-hydroxy-4-(3-methoxycarbonyl-benzyloxy)-1-L-leucylpyrrolidine (315 mg), N-methylmorpholine (350 mg), 1-(3-dimethyl-aminopropyl)-3-ethylcarbodiimide hydrochloride (199 mg), 1-hydroxybenzotriazole (132 mg) and 2-quinolinecarboxylic acid (150 mg) in dichloromethane (15 ml) followed by chromatography over silica using 3:1 ethyl acetate:hexane gave the title compound (337 mg) as a colourless gum. ¹H NMR (CDCl₃) δ: 1.05 (m, 6H), 1.8 (m, 3H), 3.65 (m, 2H), 3.8 (m, 1H), 3.95 (m, 3H), 4.0-4.25 (m, 2H), 4.35 (m, 1H), 4.55-4.8 (m, 2H), 5 (m, 1H), 7.4-7.75 (m, 3H), 7.80 (t, 1H), 7.90 (d, 1H), 8.05 (m, 2H), 8.20 (d, 1H), 8.3 (m, 2H), 8.75 (m, 1H).

(b) 3-(3-Methoxycarbonylbenzyloxy)-1-[N-(2-quinolinecarbonyl)-L-leucyl]pyrrolidin-4-one

In a manner similar to Example 1(d) reaction of 3-hydroxy-4-(3-methoxycarbonyl-benzyloxy)-1-[N-(2-quinolinecarbonyl)-L-leucyl]pyrrolidine (327 mg) and Dess-Martin's reagent (1.1 g) in dichloromethane (12 ml) followed by chromatography over silica using 2:1 hexane:ethyl acetate gave the title compound (259 mg) as a colourless oil. ¹H NMR (CDCl₃) δ: 1.05 (m, 6H), 1.8 (m, 3H), 3.5-3.8 (m, 1H), 3.93 (m, 3H), 4.0-4.5 (m, 3H), 4.5-4.8 (m, 2H), 4.85-5.2 (m, 2H), 7.45 (m, 1H), 7.5-7.7 (m, 2H), 7.80 (t, 1H), 7.90 (d 1H), 8.0 (m, 2H), 8.15 (d, 1H), 8.25 (m, 2H), 8.7 (m, 1H).

### Example 15

### Preparation of 3-(4-Nitrobenzyloxy)-1-[N-(2-quinolinecarbonyl)-L-leucyl]pyrrolidin-4-one

(a) 3-Hydroxy-4-(4-nitrobenzyloxy)-1-N-phthaloyl-L-leucyl)pyrrolidine

In a manner similar to Example 7(c) reaction of 3,4-dihydroxy-1-(N-phthaloyl-L-leucyl)-pyrrolidine (0.80 g), 60% sodium hydride in oil (0.10 g) and 4-nitrobenzyl bromide (0.64 g) in dry dimethylformamide (8 ml) followed by chromatography over silica using 3:1 ethyl acetate:hexane gave the title compound (190 mg) as a colourless oil. ¹H NMR (CDCl₃) δ: 1.0 (m, 6H), 1.55 (m, 2H), 1.75 (m, 1H), 2.3-2.7 (m, 2H), 3.4-3.8 (m, 3H), 4.05 (m, 1H), 4.35 (m, 1H), 4.6-4.8 (m, 2H), 4.95 (m, 1H), 7.48 (m, 2H), 7.75 (m, 2H), 7.85 (m, 2H), 8.2 (m, 2H).

(b) 3-Hydroxy-4-(4-nitrobenzyloxy)-1-L-leucylpyrrolidine

In a manner similar to Example 7(d) reaction of 3 hydroxy-4-(4-nitrobenzyloxy)-1-(N-phthaloyl-L-leucyl)pyrrolidine (225 mg) and hydrazine hydrate (0.12 g) in ethanol (4 ml) gave the title compound (109 mg) as a colourless oil. ¹H NMR (CDCl₃) δ: 0.95 (m, 6H), 1.3-1.5 (m, 2H), 1.8 (m, 1H), 3.4-3.9 (m, 5H), 4.1 (m, 1H), 4.4(m, 1H), 4.65-4.9 (m, 2H), 7.5 (m, 2H), 8.25 (m, 2H).

(c) 3-Hydroxy-4-(4-nitrobenzyloxy)-1-[N-(2-quinolinecarbonyl)-L-leucyl]pyrrolidine

In a manner similar to Example 1 (c) reaction of 3-hydroxy-4-(4-nitrobenzyloxy)-1-L-leucylpyrrolidine (100 mg), N-methylmorpholine (115 mg), 1-(3-dimethylamino-propyl)-3 -ethylcarbodiimide hydrochloride (65 mg), 1-hydroxybenzotriazole (44 mg) and 2-quinoline-carboxylic acid (49 mg) in dichloromethane (5 ml) followed by chromatography over silica using 3:1 ethyl acetate:hexane gave the title compound (124 mg) as a pale buff gum. ¹H NMR (CDCl₃) δ: 1.05 (m, 6H),1.6-1.9 (m, 3H), 3.55-3.9 (m, 3H), 4.05-4.3 (m, 2H), 4.45 (m, 1H), 4.65-4.85 (m, 2H), 5.05 (m, 1H), 7.4-7.7 (m, 3H), 7.78 (t, 1H), 7.87 (m, 2H), 8.2 (m, 2H), 8.3 (m, 3H), 8.75 (m, 1H).

(d) 3-(4-Nitrobenzyloxy)-1-[N-(2-quinolinecarbonyl)-L-leucyl]pyrrolidin-4-one

In a manner similar to Example 1(d) reaction of 3-hydroxy-4-(4-nitrobenzyloxy)-1-[N-(2-quinolinecarbonyl)-L-leucyl]pyrrolidine (114 mg) and Dess-Martin's reagent (0.80 g) in dichloromethane (6 ml) followed by chromatography over silica using 2:1 hexane:ethyl acetate gave the title compound (40 mg) as a colourless oil. ¹H NMR (CDCl₃) δ: 1.0 (m, 6H), 1.65-1.9 (m, 3H), 3.55-3.9 (m, 1H), 4.05-4.3 (m, 3H), 4.4-4.65 (m, 1H), 4.7-5.25 (m, 3H), 7.4-7.7 (m, 3H), 7.79 (t, 1H), 7.87 (d, 1H), 8.05-8.35 (m, 5H), 8.7 (m, 1H).

### Example 16

### Preparation of 3-(5-Methyl-3-isoxazolylmethoxy)-1-[N-(2-quinolinecarbonyl)-L-leucyl]pyrrolidin-4-one

(a) 3-Hydroxy-4-(5-methyl-3-isoxazolylmethoxy)-1-(N-phthaloyl-L-leucyl)pyrrolidine

In a manner similar to Example 7(c) reaction of 3,4-dihydroxy-1-(N-phthaloyl-L-leucyl)-pyrrolidine (0.40 g), 60% sodium hydride in oil (50 mg) and 3-bromomethyl-5-methyl-isoxazole (0.25 g) in dry dimethylformamide (5 ml) followed by chromatography over silica using 3:1 ethyl acetate:hexane gave the title compound (116 mg) as a glassy solid. ¹H NMR (CDCl₃) δ: 1.0 (m, 6H), 1.55 (m, 2H), 1.75 (m, 1H), 2.45 (m, 3H), 2.5-2.8 (m, 2H), 3.45-3.8 (m, 3H), 4.0 (m, 1H), 4.25 (m, 1H), 4.55-4.8 (m, 2H), 4.95 (m, 1H), 6.00 (t, 1H) 7.75 (m, 2H), 7.85 (m, 2H).

(b) 3-Hydroxy-4-(5-methyl-3-isoxazolylmethoxy)-1-L-leucyl-pyrrolidine

In a manner similar to Example 7(d) reaction of 3-hydroxy-4-(5-methyl-3-isoxazolyl-methoxy)-1-(N-phthaloyl-L-leucyl)pyrrolidine (106 mg) and hydrazine hydrate (60 mg) in ethanol (2.5 ml) gave the title compound (66 mg) as a colourless oil. ¹H NMR (CDCl₃) δ: 0.95 (m, 6H), 1.25-1.55 (m, 2H), 1.85 (m, 1H), 2.44 (s, 3H), 3.35-3.7 (m, 4H), 3.75 (m, 1H), 4.05 (m, 1H), 4.3 (m, 1H), 4.6-4.8 (m, 2H), 6.02 (m, 1H).

(c) 3-Hydroxy-4-(5-methyl-3-isoxazolylmethoxy)-1-[N-(2-quinolinecarbonyl)-L-leucyl]pyrrolidine

In a manner similar to Example 1(c) reaction of 3-hydroxy-4-(5-methyl-3-isoxazolyl-methoxy)-1-L-leucylpyrrolidine (56 mg), N-methylmorpholine (74 mg), 1-(3-dimethyl-aminopropyl)-3-ethylcarbodiimide hydrochloride (41 mg), 1-hydroxybenzotriazole (28 mg) and 2-quinolinecarboxylic acid (32 mg) in dichloromethane (3 ml) followed by chromatography over silica using 3:1 ethyl acetate:hexane gave the title compound (61 mg) as a pale buff gum. ¹H NMR (CDCl₃) δ: 0.95-1.1 (m, 6H), 1.6-1.9 (m, 3H), 2.45 (m, 3H), 2.75-2.95 (m, 1H), 3.5-3.7 (m, 2H), 3.8 (m, 1H), 4.0-4.2 (m, 2H), 4.3 (m, 1H), 4.55-4.8 (m ,2H), 5.0 (m, 1H), 6.02 (d, 1H), 7.61 (t, 1H), 7.77 (t, 1H), 7.86 (d, 1H), 8.15 (d, 1H), 8.25 (m, 2H), 8.75 (m, 1H).

(d) 3-(5-Methyl-3-isoxazolylmethoxy)-1-[N-(2-quinolinecarbonyl)-L-leucyl]pyrrolidin-4-one

In a manner similar to Example 1(d) reaction of 3-hydroxy-4-(5-methyl-3-isoxazolyl-methoxy)-1-[N-(2-quinolinecarbonyl)-L-leucyl]pyrrolidine (51 mg) and Dess-Martin's reagent (0.29 g) in dichloromethane (2 ml) followed by chromatography over silica using 1:1 hexane:ethyl acetate gave the title compound (37 mg) as a colourless gum. ¹H NMR (CDCl₃) δ: 0.95 (m, 6H), 1.6-1.9 (m, 3H), 2.34 (d, 3H), 3.35-3.55 (m, 1H), 3.4-4.4 (m, 3H), 4.5-4.95 (m, 3H), 5.05 (m, 9H), 5.99 (d, 1H), 7.55 (t, 1H), 7.70 (t, 1H), 7.80 (d, 1H), 8.07 (d, 1H), 8.2 (m, 2H), 8.65 (m, 1H).

### Example 17

### Preparation of 3-(3-Methoxybenzyloxy)-1-[N-(2-naphthalenecarbonyl)-L-leucyl]pyrrolidin-4-one

(a) 3-Hydroxy-4-(3-methoxybenzyloxy)-1-[N-(2-naphthalenecarbonyl)-L-leucyl]pyrrolidine

In a manner similar to Example 1(c) reaction of 3-hydroxy-4-(3-methoxybenzyloxy)-1-L-leucylpyrrolidine (228 mg), N-methylmorpholine (276 mg), 1-(3-dimethylaminopropyl)-3-ethylcarbodiimide hydrochloride (156 mg), 1-hydroxybenzotriazole (104 mg) and 2-naphthoic acid (117 mg) in dichloromethane (12 ml) followed by chromatography over silica using 4:1 ethyl acetate:hexane gave the title compound (295 mg) as a colourless gum. ¹H NMR (CDCl₃) δ: 0.95 (m, 6H), 1.45-1.9 (m, 3H), 3.5-3.7 (m, 3H), 3.84 (t, 3H), 3.95-4.2 (m, 2H), 4.35 (m, 1H), 4.5-4.7 (m, 2H), 5.05 (m, 1H), 6.9 (m, 3H), 7.1 (m, 1H) 7.3 (m, 1H), 7.55 (m, 2H), 7.9 (m, 4H), 8.35 (s, 1H).

(b) 3-(3-Methoxybenzyloxy)-1-[N-(2-naphthalenecarbonyl)-L-leucyl]pyrrolidin-4-one

In a manner similar to Example 1(d) reaction of 3-hydroxy-4-(3-methoxybenzyloxy)-1-[N-(2-naphthalenecarbonyl)-L-leucyl]pyrrolidine (280 mg) and Dess-Martin's reagent (0.48 g) in dichloromethane (7.5 ml) followed by chromatography over silica using 2:1 hexane:ethyl acetate gave the title compound (246 mg) as a colourless gum. ¹H NMR (CDCl₃) δ: 1.05 (m, 6H), 1.5-1.9 (m, 3H), 3.45-3.75 (m, 1H), 3.83 (m, 3H), 3.9-4.05 (m, 1H), 4.05-4.2 (m, 1H), 4.2-4.4 (m, 1H), 4.4-4.7 (m, 2H), 4.75-5.05 (m, 1H), 5.15 (m, 1H), 6.95 (m, 4H), 7.25 (m, 1H), 7.55 (m, 2H), 7.85 (m, 4H), 8.3 (s, 1H).

### Example 18

### Preparation of 3-(4-Methoxyphenoxy)-1-(N-carbobenzyloxy-L-leucyl)pyrrolidin-4-one

(a) N-(carbobenzyloxy-L-leucyl)-3-pyrroline

In a manner similar to Example 1(c) reaction of 3-pyrroline (0.21 g), N-methylmorpholine (1.66 g), 1-(3-dimethylaminopropyl)-3-ethylcarbodiimide hydrochloride (0.70 g), 1-hydroxybenzotriazole (0.47 g) and N-carbobenzyloxy-L-leucine (0.80 g) in dichloromethane (10 ml) followed by chromatography over silica using 2:3 ethyl acetate:hexane gave the title compound (0.70 g) as a white solid. ¹H NMR (CDC1₃) δ: 0.97 (d of d, 6H), 1.35-1.65 (m, 2H), 1.75 (m, 1H), 4.1-4.4 (m, 3H), 4.5 (m, 2H), 5.08 (s, 2H), 5.45 (d, 1H), 5.85 (m, 2H), 7.34 (s, 5H).

(b) N-(carbobenzyloxy-L-leucyl)-3,4-epoxypyrrolidine

To a solution of N-(carbobenzyloxy-L-leucyl)-3-pyrroline (0.64 g) in dichloromethane (20 ml) was added m-chloroperoxybenzoic acid (1.45 g) and the solution stirred for 48 hours. The mixture was filtered and the filtrate washed successively with saturated NaH₂SO₃ in 2N sodium hydroxide solution, saturated sodium bicarbonate solution and water, dried (magnesium sulphate) and evaporated down under reduced pressure to give the title compound (0.31 g) as a colourless oil. ¹H NMR (CDCl₃) δ: 0.95 (d of d, 6H), 1.3-1.65 (m, 2H), 1.7 (m, 1H), 3.3-3.6 (m, 2H), 3.7-3.95 (m, 3H), 4.0-4.15 (m, 1H), 4.3-4.5 (m, 1H), 5.07 (s, 2H), 5.4 (d of d, 1H), 7.34 (s, 5H).

(c) 3-hydroxy-4-(4-methoxyphenoxy)-1-(N-carbobenzyloxy-L-leucyl)pyrrolidine

To a solution of 4-methoxyphenol (150 mg) in dry tetrahydrofuran (0.5 ml) was added 1M potassium tert-butoxide in tetrahydrofuran (1.8 ml) followed by a solution of N-(carbobenzyloxy-L-leucyl)-3,4-epoxypyrrolidine (195 mg) in dry tetrahydrofuran (1.5 ml) and the mixture stirred at 50° for 16 hours then under reflux for 3 hours. Solvent was evaporated off under reduced pressure and the residue dissolved in dichloromethane (8 ml). The solution was washed successively with water and brine, dried (magnesium sulphate) 25 and evaporated down under reduced pressure. Chromatography over silica using 1:1ethyl acetate:hexane gave the title compound (88 mg) as a pale buff gum. ¹H NMR (CDCl₃) δ: 0.95 (m, 6H), 1.3-1.6 (m, 3H), 3.3-3.75 (m, 2H), 3.77 (s, 3H), 3.85-4.05 (m, 1H), 4.3-4.7 (m, 3H), 5.05 (m, 2H), 5.3-5.55 (m, 1H), 6.82 (m, 4H), 7.33 (s, 5H).

(d) 3-(4-Methoxyphenoxy)-1-(N-carbobenzyloxy-L-leucyl)pyrrolidinone

In a manner similar to Example 1(d) reaction of 3-hydroxy-4-(4-methoxyphenoxy)-1-(N-carbobenzyloxy-L-leucyl)pyrrolidine (78 mg) and Dess-Martin's reagent (300 mg) in dichloromethane (3 ml) followed by chromatography over silica using 2:1 hexane:ethyl acetate gave the title compound (45 mg) as a colourless oil. ¹H NMR (CDC1₃) δ0.95 (m, 6H), 1.4-1.8 (m, 3H), 3.5-3.7 (m, 1H), 3.78 (d, 3H), 3.9-4.1 (m, 1H), 4.2.-4.55 (m, 2H), 4.55-4.7 (m, 1H), 4.7-4.9 (m, 1H), 5.09 (s, 2H), 5.4 (m, 1H), 6.75-6.95 (m, 4H), 7.34 (s, 5H).

What is claimed is:

1. A compound according to formula (I): 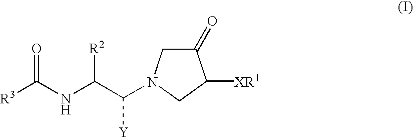
wherein:
X is selected from the group consisting of oxygen, sulfur, SO, and SO₂;
Y is selected from the group consisting of H₂ and oxygen; where if Y is H_{2,} then the - - - bond represents two single bonds and where if Y is O, then the - - - bond represents a double bond;
R¹ is selected from the group consisting of hydrogen, C₁₋₆ alkyl, C₂₋₆ alkenyl, C₂₋₆ alkynyl, C₃₋₆ cycloalkyl-C₀₋₆ alkyl, Ar-C₀₋₆ alkyl, Het-C₀₋₆ alkyl, (CH₂)₀₋₆CO₂R″, and (CH₂)₀₋₆Ar;
R″ is selected from the group consisting of hydrogen, C₁₋₆ alkyl, C₂₋₆ alkenyl, C₂₋₆ alkynyl, C₃₋₆ cycloalkyl-C₀₋₆ alkyl, Ar-C₀₋₆ alkyl, and Het-C₀₋₆ alkyl;
R² is selected from the group consisting of H, C₁₋₆ alkyl, C₂₋₆ alkenyl, C₂₋₆ alkynyl, C₃₋₆ cycloalkyl-C₀₋₆ alkyl, Ar-C₀₋₆ alkyl, and Het-C₀₋₆ alkyl;
R³ is selected from the group consisting of H, C₁₋₆ alkyl, C₂₋₆ alkenyl, C₂₋₆ alkynyl, C₃₋₆ cycloalkyl-C₀₋₆ alkyl, Ar-C₀₋₆ alkyl, and Het-C₀₋₆ alkyl;
C ₁₋₆ alkyl is selected from the group consisting of substituted and unsubstituted methyl, ethyl, n-propyl, isopropyl, n-butyl, isobutyl, t-butyl, pentyl, n-pentyl, isopentyl, neopentyl hexyl and aliphatic isomers thereof;
C₃₋₆ cycloalkyl is selected from the group consisting of substituted and unsubstituted cyclopropane, cyclobutane, cyclopentane, and cyclohexane;
C₂₋₆ alkenyl is an alkyl group of 2 to 6 carbons, wherein a carbon-carbon single bond is replaced by a carbon-carbon double bond;
C₂₋₆ alkynyl is an alkyl group of 2 to 6 carbons, wherein one carbon-carbon single bond is replaced by a carbon-carbon triple bond;
Ar is selected from the group consisting of phenyl or naphthyl; or phenyl or naphthyl substituted by one or more of Ph-C₀₋₆ alkyl, Het-C₀₋₆ alkyl, C₁₋₆ alkoxy, Ph-C₀₋₆ alkoxy, Het-C₀₋₆ alkoxy, OH, (CH₂)₀₋₆CO₂R″, where R″ is as defined above, (CH₂)₁₋₆NR′R′, O(CH₂)₁₋₆NR′R′; or phenyl or naphthyl substituted by one to three moieties selected from C₁₋₄alkyl, OR′, N(R′)₂, SR′, CF₃, NO₂, CN, CO₂R′, CON(R′), F, Cl, Br and I, or substituted by a methylenedioxy group; wherein each R′ independently is H, C₁₋₆ alkyl, Ar-C₀₋₆ alkyl, or Het-C₀₋₆ alkyl;
Het is a stable 5- to 7-membered monocyclic or a stable 7- to 10-membered bicyclic heterocyclic ring, which is either saturated or unsaturated, and which consists of carbon atoms and from one to four heteroatoms selected from the group consisting of N, O and S;
and pharmaceutically acceptable salts, hydrates, and isomers thereof.

2. The compound according to claim 1, wherein X is O.

3. The compound according to claim 1, wherein Y is O.

4. The compound according to claim 1, wherein R¹ is selected from the group consisting of

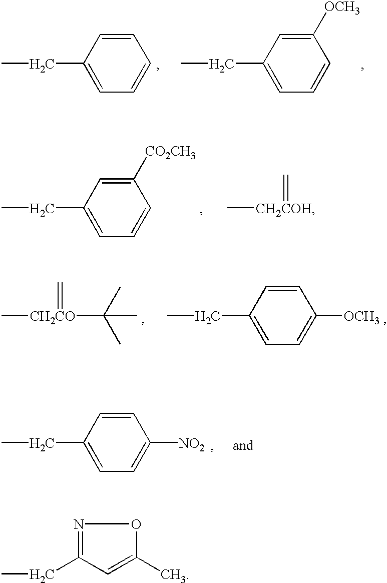

5. The compound according to claim 1, wherein R² is isobutyl or a substituted isobutyl.

6. The compound according to claim 1, wherein R³ is selected from the group consisting of

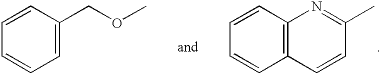

7. The compound according to claim 1, wherein said Het group is substituted with one to three moieties selected from the group consisting of C₁₋₄ alkyl, OR′, N(R′)₂, SR′, CF₃, NO₂, CN, CO₂R′, CON(R′), F, Cl, Br, and I, wherein each R′ independently is H, C₁₋₄ alkyl, Ar-C₀₋₆ or Het-C₀₋₆ alkyl.

8. The compound according to claim 1, wherein X is O, Y is O, R¹ is selected from the group consisting of 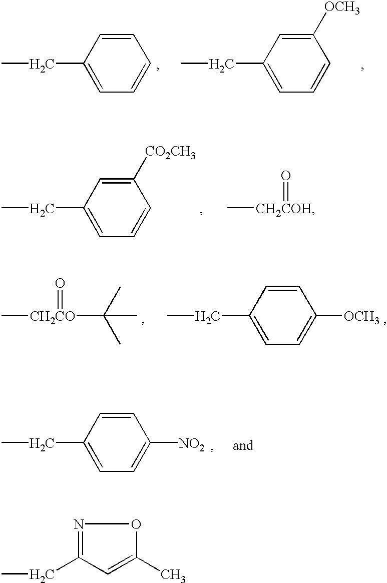
R² is isobutyl; and
R³ is selected from the group consisting of

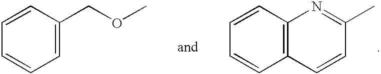

9. The compound according to claim 1, which is selected from the group consisting of:
3-Benzyloxy-1-(N-carbobenzyloxy-L-leucyl)pyrrolidin-4-one;
3-Benzylthio-1-(N-carbobenzyloxy-L-leucyl)pyrrolidin-4-one;
3-Benzylsulfinyl-1-(N-carbobenzyloxy-L-leucyl)pyrrolidin-4-one;
3-Benzylsulfonyl-1-(N-carbobenzyloxy-L-leucyl)pyrrolidin-4-one;
1-[2-(Benzyloxycarbonylamino)-4-methylpentyl]-3-benzylthiopyrrolidin-4-one;
3-Benzylthio-1-[N-(2-quinolinecarbonyl)-L-leucyl]pyrrolidin-4-one;
3-tert-Butoxycarbonylmethoxy-1-(N-carbobenzyloxy-L-leucyl)pyrrolidin-4-one;
3-(3-Methoxybenzyloxy)-1-(N-carbobenzyloxy-L-leucyl)pyrrolidin-4-one;
3-(3-Methoxycarbonylbenzyloxy)-1-(N-carbobenzyloxy-L-leucyl)pyrrolidin-4-one;
3-tert-Butoxycarbonylmethoxy-1-[N-(2-quinolinecarbonyl)-L-leucyl]pyrrolidin-4-one;
1-[N-(2-Quinolinecarbonyl)-L-leucyl]-3-oxo-4-pyrrolidineoxyacetic acid;
3-(3-Methoxybenzyloxy)-1-[N-(2-quinolinecarbonyl)-L-leucyl]pyrrolidin-4-one;
3-(4-Methoxybenzyloxy)-1-[N-(2-quinolinecarbonyl)-L-leucyl]pyrrolidin-4-one;
3-(3-Methoxycarbonylbenzyloxy)-1-[N-(2-quinolinecarbonyl)-L-leucyl]pyrrolidin-4-one;
3-(4-Nitrobenzyloxy)-1-[N-(2-quinolinecarbonyl)-L-leucyl]pyrrolidin-4-one;
3-(5-Methyl-3-isoxazolylmethoxy)-1-[N-(2-quinolinecarbonyl)-L-leucyl]pyrrolidin-4-one;
3-(3-Methoxybenzyloxy)-1-[N-(2-naphthalenecarbonyl)-L-leucyl]pyrrolidin-4-one; and
3-(4-Methoxyphenoxy)-1-(N-carbobenzyloxy-L-leucyl)pyrrolidin-4-one and pharmaceutically acceptable salts, hydrates and isomers thereof.

10. A pharmaceutically effective composition comprising a compound according to claim 1 and a pharmaceutically acceptable carrier, diluent or excipient.

11. A method of inhibiting a cysteine protease which comprises administering to a patient in need thereof an effective amount of a compound according to claim 1.

12. A method according to claim 11, wherein the cysteine protease is cathepsin K.

13. A method of inhibiting bone loss which comprises administering to a patient in need thereof an effective amount of a compound according to claim 1.

14. A method of treating osteoporosis which comprises administering to a patient in need thereof an effective amount of a compound according to claim 1.

15. A method of treating gingival or peridontal disease which comprises administering a patient in need thereof an effective amount of a compound according to claim 1.

16. A method of treating a disease characterized by excessive cartilage or matrix degradation which comprises administering to a patient in need thereof an effective amount of a compound according to claim 1.

17. A method according to claim 16, wherein said disease is osteoarthritis or rheumatoid arthritis.

18. A compound selected from the group consisting of:
3-Benzyloxy-4-hydroxy-1-(N-carbobenzyloxy-L-leucyl)pyrrolidine;
3-Benzylthio-4-hydroxy-1-(N-carbobenzyloxy-L-leucyl)pyrrolidine;
1-[2-(Benzyloxycarbonylamino)-4-methyl-pentyl]-3-benzylthio-4-hydroxypyrrolidine;
3-Benzylthio-4-hydroxy-1-[N-(2-quinolinecarbonyl)-L-leucyl]pyrrolidine;
3-tert-(Butoxycarbonylmethoxy-4-hydroxy-1-(N-carbobenzyloxy-L-leucyl)pyrrolidine;
3-Hydroxy-4-(3-methoxybenzyloxy)-1-(N-carbo-benzyloxy-L-leucyl)pyrrolidine;
3-Hydroxy-4-(3-methoxy-carbonylbenzyloxy)-1-(N-carbobenzyloxy-L-leucyl)pyrrolidine;
3-tert-Butoxycarbonylmethoxy-1-[N-(2-quinolinecarbonyl)-L-leucyl]pyrrolidine;
3-Hydroxy-4-(3-methoxycarbonylbenzyloxy)-1-[N-(2-quinolinecarbonyl)-L-leucyl]pyrrolidine;
3-Hydroxy-4-(4-nitrobenzyloxy)-1-[N-(2-quinolinecarbonyl)-L-leucyl]pyrrolidine;
3-Hydroxy-4-(5-Methyl-3-isoxazolylmethoxy)-1-[N-(2-quinolinecarbonyl)-L-leucyl]pyrrolidine;
3-Hydroxy-4-(3-methoxybenzyloxy)-1-[N-(2-naphthalenecarbonyl)-L-leucyl]pyrrolidine;
3-hydroxy-4-(4-methoxyphenoxy)-1-(N-carbobenzyloxy-L-leucyl)pyrrolidine;
1-hydroxy-7-azabenzotriazole; and
1,1,1-triacetoxy-1,1-dihydro-1,2-benziodoxol-3(1H)-one.

19. A process of producing an pyrrolidinone cathepsin K inhibitor comprising the step of converting a compound selected from the group consisting of:
3-Benzyloxy-4-hydroxy-1-(N-carbobenzyloxy-L-leucyl)pyrrolidine;
3-Benzylthio-4-hydroxy-1-(N-carbo-benzyloxy-L-leucyl)pyrrolidine;
1-[2-(Benzyloxycarbonylamino)-4-methylpentyl]-3-benzylthio-4-hydroxypyrrolidine;
3-Benzylthio-4-hydroxy-1-[N-(2-quinolinecarbonyl)-1-leucyl]pyrrolidine;
3-tert-Butoxycarbonylmethoxy-4-hydroxy-1-(N-carbobenzyloxy-L-leucyl)pyrrolidine;
3-Hydroxy-4-(3-methoxybenzyloxy)-1-(N-carbo-benzyloxy-L-leucyl)pyrrolidine;
3-Hydroxy-4-(3-methoxy-carbonylbenzyloxy)-1-(N-carbobenzyloxy-L-leucyl)pyrrolidine;
3-tert-Butoxycarbonylmethoxy-1-[N-(2-quinolinecarbonyl)-L-leucyl]pyrrolidine;
3-Hydroxy-4-(3-methoxycarbonylbenzyloxy)-1-[N-(2-quinolinecarbonyl)-L-leucyl]pyrrolidine;
3-Hydroxy-4-(4-nitrobenzyloxy)-1-[N-(2-quinolinecarbonyl)-L-leucyl]pyrrolidine;
3-Hydroxy-4-(5-Methyl-3-isoxazolylmethoxy)-1-[N-(2-quinolinecarbonyl)-L-leucyl]pyrrolidine;
3-Hydroxy-4-(3-methoxybenzyloxy)-1-[N-(2-naphthalenecarbonyl)-L-leucyl]pyrrolidine; and
3-hydroxy-4-(4-methoxyphenoxy)-1-(N-carbobenzyloxy-L-leucyl)pyrrolidine into an pyrrolidinone cathepsin K inhibitor.

20. The process according to claim 19, wherein said pyrrolidinone cathepsin K inhibitor is a compound having the formula (I): 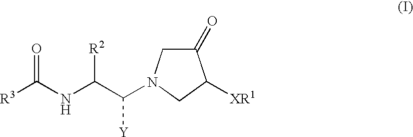
wherein:
X is selected from the group consisting of oxygen, sulfur, SO, and SO₂;
Y is selected from the group consisting of H₂ and oxygen; where if Y is H₂, then the - - - bond represents two single bonds and where if Y is O, then the - - - bond represents a double bond;
R′ is selected from the group consisting of hydrogen, C₁₋₆alkyl, C₂₋₆alkenyl, C₂₋₆alkynyl, C₃₋₆ cycloalkyl-C₀₋₆ alkyl, Ar-C₀₋₆ alkyl, Het-C₀₋₆ alkyl, (CH₂)₀₋₆CO₂R″, and (CH₂)₀₋₆Ar; R″ is selected from the group consisting of hydrogen, C₁₋₆ alkyl, C₂₋₆ alkenyl, C₂₋₆ alkynyl, C₃₋₆ cycloalkyl-C₀₋₆ alkyl, Ar-C₀₋₆ alkyl, and Het-C₀₋₆ alkyl;
R² is selected from the group consisting of H, C₁₋₆ alkyl, C₂₋₆ alkenyl, C₂₋₆ alkynyl, C₃₋₆ cycloalkyl-C₀₋₆ alkyl, Ar-C₀₋₆ alkyl, and Het-C₀₋₆ alkyl;
R³ is selected from the group consisting of H, C₁₋₆ alkyl, C₂₋₆ alkenyl, C₂₋₆ alkynyl, C₃₋₆ cycloalkyl-C₀₋₆ alkyl, Ar-C₀₋₆ alkyl, and Het-C₀₋₆ alkyl;
C₁₋₆ alkyl is selected from the group consisting of substituted and unsubstituted methyl, ethyl, n-propyl, isopropyl, n-butyl, isobutyl, t-butyl, pentyl, n-pentyl, isopentyl, neopentyl hexyl and aliphatic isomers thereof;
C₃₋₆ cycloalkyl is selected from the group consisting of substituted and unsubstituted cyclopropane, cyclobutane, cyclopentane, and cyclohexane;
C₂₋₆ alkenyl is an alkyl group of 2 to 6 carbons, wherein a carbon-carbon single bond is replaced by a carbon-carbon double bond;
C₂₋₆ alkynyl is an alkyl group of 2 to 6 carbons, wherein one carbon-carbon single bond is replaced by a carbon-carbon triple bond;
Ar is selected from the group consisting of phenyl or naphthyl; or phenyl or naphthyl substituted by one or more of Ph-C₀₋₆ alkyl, Het-C₀₋₆ alkyl, C₁₋₆ alkoxy, Ph-C₀₋₆ alkoxy, Het-C₀₋₆ alkoxy, OH, (CH₂)₀₋₆CO₂R″, where R″ is as defined above, (CH₂)₁₋₆NR′R′, O(CH₂)₁₋₆NR′R′; or phenyl or naphthyl substituted by one to three moieties selected from C₁₋₄alkyl, OR′, N(R′)₂, SR′, CF₃, NO₂, CN, CO₂R′, CON(R′), F, Cl, Br and I, or substituted by a methylenedioxy group; wherein each R′ independently is H, C₁₋₆ alkyl, Ar-C₀₋₆ alkyl, or Het-C₀₋₆ alkyl;
Het is a stable 5- to 7-membered monocyclic or a stable 7- to 10-membered bicyclic heterocyclic ring, which is either saturated or unsaturated, and which consists of carbon atoms and from one to four heteroatoms selected from the group consisting of N, O, and S;
and pharmaceutically acceptable salts, hydrates, and isomers thereof.

21. The process according to claim 20, wherein said compound is selected from the group consisting of:
3-Benzyloxy-1-(N-carbobenzyloxy-L-leucyl)pyrrolidin-4-one;
3-Benzylthio-1-(N-carbobenzyloxy-L-leucyl)pyrrolidin-4-one;
3-Benzylsulfinyl-1-(N-carbobenzyloxy-L-leucyl)pyrrolidin-4-one;
3-Benzylsulfonyl-1-(N-carbobenzyloxy-L-leucyl)pyrrolidin-4-one;
1-[2-(Benzyloxycarbonylamino)-4-methylpentyl]-3-benzylthiopyrrolidin-4-one;
3-Benzylthio-1-[N-(2-quinolinecarbonyl)-L-leucyl]pyrrolidin-4-one;
3-tert-Butoxycarbonylmethoxy-1-(N-carbobenzyloxy-L-leucyl)pyrrolidin-4-one;
3-(3-Methoxybenzyloxy)-1-(N-carbobenzyloxy-L-leucyl)pyrrolidin-4-one;
3-(3-Methoxycarbonylbenzyloxy)-1-(N-carbobenzyloxy-L-leucyl)pyrrolidin-4-one;
3-tert-Butoxycarbonylmethoxy-1-[N-(2-quinolinecarbonyl)-L-leucyl]pyrrolidin-4-one;
1-[N-(2-Quinolinecarbonyl)-L-leucyl]-3-oxo-4-pyrrolidineoxyacetic acid;
3-(3-Methoxybenzyloxy)-1-[N-(2-quinolinecarbonyl)-L-leucyl]pyrrolidin-4-one;
3-(4-Methoxybenzyloxy)-1-[N-(2-quinolinecarbonyl)-L-leucyl]pyrrolidin-4-one;
3-(3-Methoxycarbonylbenzyloxy)-1-[N-(2-quinolinecarbonyl)-L-leucyl]pyrrolidin-4-one;
3-(4-Nitrobenzyloxy)-1-[N-(2-quinotinecarbonyl)-L-leucyl]pyrrolidin-4-one;
3-(5-Methyl-3-isoxazolylmethoxy)-1-[N-(2-quinolinecarbonyl)-L-leucyl]pyrrolidin 4-one;
3-(3-Methoxybenzyloxy)-1-[N-(2-naphthalenecarbonyl)-L-leucyl]pyrrolidin-4-one; and
3-(4-Methoxyphenoxy)-1-(N-carbobenzyloxy-L-leucyl)pyrrolidin-4-one and pharmaceutically acceptable salts, hydrates and isomers thereof.
